SEQUENCE LISTING (87 sequences)

SEQ ID NO: 1 - protein, 5202 aa, Artificial Sequence
  polypeptide fragment
MNCPVLSLGSGFLFQVIEMLIFAYFASISLTESRGLFPRLENVGAFKKVSIVPTQAVCGLPDRSTFCHSSAAAESIQFCTQRFCIQDCPYRSSHPTYTALFSAGLSSCITPDKNDLHPNAHSNSASFIFGNHKSCFSSPPSPKLMASFTLAVWLKPEQQGVMCVIEKTVDGQIVFKLTISEKETMFYYRTVNGLQPPIKVMTLGRILVKKWIHLSVQVHQTKISFFINGVEKDHTPFNARTLSGSITDFASGTVQIGQSLNGLEQFVGRMQDFRLYQVALTNREILEVFSGDLLRLHAQSHCRCPGSHPRVHPLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQEFVKATQIRFHFHGQYYTTETAVNLRHRYYAVDEITISGRCQCHGHADNCDTTSQPYRCLCSQESFTEGLHCDRCLPLYNDKPFRQGDQVYAFNCKPCQCNSHSKSCHYNISVDPFPFEHFRGGGGVCDDCEHNTTGRNCELCKDYFFRQVGADPSAIDVCKPCDCDTVGTRNGSILCDQIGGQCNCKRHVSGRQCNQCQNGFYNLQELDPDGCSPCNCNTSGTVDGDITCHQNSGQCKCKANVIGLRCDHCNFGFKFLRSFNDVGCEPCQCNLHGSVNKFCNPHSGQCECKKEAKGLQCDTCRENFYGLDVTNCKACDCDTAGSLPGTVCNAKTGQCICKPNVEGRQCNKCLEGNFYLRQNNSFLCLPCNCDKTGTINGSLLCNKSTGQCPCKLGVTGLRCNQCEPHRYNLTIDNFQHCQMCECDSLGTLPGTICDPISGQCLCVPNRQGRRCNQCQPGFYISPGNATGCLPCSCHTTGAVNHICNSLTGQCVCQDASIAGQRCDQCKDHYFGFDPQTGRCQPCNCHLSGALNETCHLVTGQCFCKQFVTGSKCDACVPSASHLDVNNLLGCSKTPFQQPPPRGQVQSSSAINLSWSPPDSPNAHWLTYSLLRDGFEIYTTEDQYPYSIQYFLDTDLLPYTKYSYYIETTNVHGSTRSVAVTYKTKPGVPEGNLTLSYIIPIGSDSVTLTWTTLSNQSGPIEKYILSCAPLAGGQPCVSYEGHETSATIWNLVPFAKYDFSVQACTSGGCLHSLPITVTTAQAPPQRLSPPKMQKISSTELHVEWSPPAELNGIIIRYELYMRRLRSTKETTSEESRVFQSSGWLSPHSFVESANENALKPPQTMTTITGLEPYTKYEFRVLAVNMAGSVSSAWVSERTGESAPVFMIPPSVFPLSSYSLNISWEKPADNVTRGKVVGYDINMLSEQSPQQSIPMAFSQLLHTAKSQELSYTVEGLKPYRIYEFTITLCNSVGCVTSASGAGQTLAAAPAQLRPPLVKGINSTTIHLRWFPPEELNGPSPIYQLERRESSLPALMTTMMKGIRFIGNGYCKFPSSTHPVNTDFTGIKASFRTKVPEGLIVFAASPGNQEEYFALQLKKGRLYFLFDPQGSPVEVTTTNDHGKQYSDGKWHEIIAIRHQAFGQITLDGIYTGSSAILNGSTVIGDNTGVFLGGLPRSYTILRKDPEIIQKGFVGCLKDVHFMKNYNPSAIWEPLDWQSSEEQINVYNSWEGCPASLNEGAQFLGAGFLELHPYMFHGGMNFEISFKFRTDQLNGLLLFVYNKDGPDFLAMELKSGILTFRLNTSLAFTQVDLLLGLSYCNGKWNKVIIKKEGSFISASVNGLMKHASESGDQPLVVNSPVYVGGIPQELLNSYQHLCLEQGFGGCMKDVKFTRGAVVNLASVSSGAVRVNLDGCLSTDSAVNCRGNDSILVYQGKEQSVYEGGLQPFTEYLYRVIASHEGGSVYSDWSRGRTTGAAPQSVPTPSRVRSLNGYSIEVTWDEPVVRGVIEKYILKAYSEDSTRPPRMPSASAEFVNTSNLTGILTGLLPFKNYAVTLTACTLAGCTESSHALNISTPQEAPQEVQPPVAKSLPSSLLLSWNPPKKANGIITQYCLYMDGRLIYSGSEENYIVTDLAVFTPHQFLLSACTHVGCTNSSWVLLYTAQLPPEHVDSPVLTVLDSRTIHIQWKQPRKISGILERYVLYMSNHTHDFTIWSVIYNSTELFQDHMLQYVLPGNKYLIKLGACTGGGCTVSEASEALTDEDIPEGVPAPKAHSYSPDSFNVSWTEPEYPNGVITSYGLYLDGILIHNSSELSYRAYGFAPWSLHSFRVQACTAKGCALGPLVENRTLEAPPEGTVNVFVKTQGSRKAHVRWEAPFRPNGLLTHSVLFTGIFYVDPVGNNYTLLNVTKVMYSGEETNLWVLIDGLVPFTNYTVQVNISNSQGSLITDPITIAMPPGAPDGVLPPRLSSATPTSLQVVWSTPARNNAPGSPRYQLQMRSGDSTHGFLELFSNPSASLSYEVSDLQPYTEYMFRLVASNGFGSAHSSWIPFMTAEDKPGPVVPPILLDVKSRMMLVTWQHPRKSNGVITHYNIYLHGRLYLRTPGNVTNCTVMHLHPYTAYKFQVEACTSKGCSLSPESQTVWTLPGAPEGIPSPELFSDTPTSVIISWQPPTHPNGLVENFTIERRVKGKEEVTTLVTLPRSHSMRFIDKTSALSPWTKYEYRVLMSTLHGGTNSSAWVEVTTRPSRPAGVQPPVVTVLEPDAVQVTWKPPLIQNGDILSYEIHMPDPHITLTNVTSAVLSQKVTHLIPFTNYSVTIVACSGGNGYLGGCTESLPTYVTTHPTVPQNVGPLSVIPLSESYVVISWQPPSKPNGPNLRYELLRRKIQQPLASNPPEDLNRWHNIYSGTQWLYEDKGLSRFTTYEYMLFVHNSVGFTPSREVTVTTLAGLPERGANLTASVLNHTAIDVRWAKPTVQDLQGEVEYYTLFWSSATSNDSLKILPDVNSHVIGHLKPNTEYWIFISVFNGVHSINSAGLHATTCDGEPQGMLPPEVVIINSTAVRVIWTSPSNPNGVVTEYSIYVNNKLYKTGMNVPGSFILRDLSPFTIYDIQVEVCTIYACVKSNGTQITTVEDTPSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRGLSKAVRARTKEDVPQGVSPPTWTKIDNLEDTIVLNWRKPIQSNGPIIYYILLRNGIERFRGTSLSFSDKEGIQPFQEYSYQLKACTVAGCATSSKVVAATTQGVPESILPPSITALSAVALHLSWSVPEKSNGVIKEYQIRQVGKGLIHTDTTDRRQHTVTGLQPYTNYSFTLTACTSAGCTSSEPFLGQTLQAAPEGVWVTPRHIIINSTTVELYWSLPEKPNGLVSQYQLSRNGNLLFLGGSEEQNFTDKNLEPNSRYTYKLEVKTGGGSSASDDYIVQTPMSTPEEIYPPYNITVIGPYSIFVAWIPPGILIPEIPVEYNVLLNDGSVTPLAFSVGHHQSTLLENLTPFTQYEIRIQACQNGSCGVSSRMFVKTPEAAPMDLNSPVLKALGSACIEIKWMPPEKPNGIIINYFIYRRPAGIEEESVLFVWSEGALEFMDEGDTLRPFTLYEYRVRACNSKGSVESLWSLTQTLEAPPQDFPAPWAQATSAHSVLLNWTKPESPNGIISHYRVVYQERPDDPTFNSPTVHAFTVKGTSHQAHLYGLEPFTTYRIGVVAANHAGEILSPWTLIQTLESSPSGLRNFIVEQKENGRALLLQWSEPMRTNGVIKTYNIFSDGFLEYSGLNRQFLFRRLDPFTLYTLTLEACTRAGCAHSAPQPLWTDEAPPDSQLAPTVHSVKSTSVELSWSEPVNPNGKIIRYEVIRRCFEGKAWGNQTIQADEKIVFTEYNTERNTFMYNDTGLQPWTQCEYKIYTWNSAGHTCSSWNVVRTLQAPPEGLSPPVISYVSMNPQKLLISWIPPEQSNGIIQSYRLQRNEMLYPFSFDPVTFNYTDEELLPFSTYSYALQACTSGGCSTSKPTSITTLEAAPSEVSPPDLWAVSATQMNVCWSPPTVQNGKITKYLVRYDNKESLAGQGLCLLVSHLQPYSQYNFSLVACTNGGCTASVSKSAWTMEALPENMDSPTLQVTGSESIEITWKPPRNPNGQIRSYELRRDGTIVYTGLETRYRDFTLTPGVEYSYTVTASNSQGGILSPLVKDRTSPSAPSGMEPPKLQARGPQEILVNWDPPVRTNGDIINYTLFIRELFERETKIIHINTTHNSFGMQSYIVNQLKPFHRYEIRIQACTTLGCASSDWTFIQTPEIAPLMQPPPHLEVQMAPGGFQPTVSLLWTGPLQPNGKVLYYELYRRQIATQPRKSNPVLIYNGSSTSFIDSELLPFTEYEYQVWAVNSAGKAPSSWTWCRTGPAPPEGLRAPTFHVISSTQAVVNISAPGKPNGIVSLYRLFSSSAHGAETVLSEGMATQQTLHGLQAFTNYSIGVEACTCFNCCSKGPTAELRTHPAPPSGLSSPQIGTLASRTASFRWSPPMFPNGVIHSYELQFHVACPPDSALPCTPSQIETKYTGLGQKASLGGLQPYTTYKLRVVAHNEVGSTASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 2 - DNA, 15606 bp, Artificial Sequence
  polynucleotide fragment
atgaattgcccagttctttcattgggctctggcttcttgtttcaggtcattgaaatgttgatctttgcctattttgcttcaatatccttgactgagtcacgaggtcttttcccaaggctggagaacgtgggagctttcaagaaagtttccatcgtgccaacccaagcagtatgtggactcccagaccgaagcactttttgtcacagctctgctgctgctgaaagtattcagttctgtacccagcggttttgtattcaggattgcccatacagatcttcacaccctacctacactgcccttttctcagcaggcctcagtagctgcatcacaccagacaagaatgatctgcatcctaacgcccatagcaattctgcaagttttatttttggaaatcacaagagctgcttttcttctcctccttctccaaagctgatggcatcatttaccttagctgtatggctgaaacctgagcaacaaggtgtaatgtgtgttatagaaaagacagtagatgggcagattgtgttcaaacttacaatatctgagaaagagaccatgttttattatcgcacagtaaatggtttgcaacctccaataaaagtaatgacactggggagaattcttgtgaagaaatggattcatcttagtgtgcaggtgcatcagacaaaaatcagcttctttatcaatggcgtggagaaggatcatacacctttcaatgcaagaactctaagtggttcaattacagattttgcatctggtactgtgcaaataggacagagtttaaatggtttagagcagtttgtcggaagaatgcaagattttcgattataccaagtggcacttacaaacagagagattctggaagtcttctctggagatcttctcagattgcatgcccaatcacattgccgttgccctggcagccacccgcgggtccaccctttggcacagcggtactgcattcctaatgatgcaggagacacagctgataatagagtgtcacggttgaatcctgaagcccatcctctctcttttgtcaatgataatgatgttggtacttcatgggtttcaaatgtgtttacaaacattacacagcttaatcaaggagtgactatttcagttgatttggaaaatggacagtatcaggtgttttatattatcattcagttctttagtccacaaccaacggaaataaggattcaaaggaagaaggaaaatagtttagattgggaggactggcaatattttgccaggaattgtggtgcttttggaatgaaaaacaatggagatttggaaaaacctgattctgtcaactgtcttcagctttccaattttactccatattcccgtggcaatgtcacatttagcatcctgacacctggaccaaattatcgtcctggatacaataacttctataataccccatctcttcaagagttcgtaaaagccacgcaaataaggtttcattttcatgggcagtactatacaactgagactgctgttaacctcagacacagatattatgcagtggacgaaatcaccattagtgggagatgtcagtgccatggtcatgccgataactgcgacacaacaagccagccatatagatgcctctgctcccaggagagcttcactgaaggacttcattgtgatcgctgcttgcctctttataatgacaagcctttccgccaaggtgatcaagtttacgctttcaattgtaaaccttgtcaatgcaacagccattccaaaagctgccattacaacatctctgtagacccatttccttttgagcacttcagagggggaggaggagtttgtgatgattgtgagcataacactacaggaaggaactgtgagctgtgcaaggattactttttccgacaagttggtgcagatccttcggccatagatgtttgcaaaccctgtgactgtgatacagttggcactagaaatggtagcattctttgtgatcagattggaggacagtgtaattgtaagagacacgtgtctggcaggcagtgcaatcagtgccagaatggattctacaatctacaagagttggatcctgatggctgcagtccctgtaactgcaatacctctgggacagtggatggagatattacctgtcaccaaaattcaggccagtgcaagtgcaaagcaaacgttattgggcttaggtgtgatcattgcaattttggatttaaatttctccgaagctttaatgatgttggatgtgagccctgccagtgtaacctccatggctcagtgaacaaattctgcaatcctcactctgggcagtgtgagtgcaaaaaagaagccaaaggacttcagtgtgacacctgcagagaaaacttttatgggttagatgtcaccaattgtaaggcctgtgactgtgacacagctggatccctccctgggactgtctgtaatgctaagacagggcagtgcatctgcaagcccaatgttgaagggagacagtgcaataaatgtttggagggaaacttctacctacggcaaaataattctttcctctgtctgccttgcaactgtgataagactgggacaataaatggctctctgctgtgtaacaaatcaacaggacaatgtccttgcaaattaggggtaacaggtcttcgctgtaatcagtgtgagcctcacaggtacaatttgaccattgacaattttcaacactgccagatgtgtgagtgtgattccttggggacattacctgggaccatttgtgacccaatcagtggccagtgcctgtgtgtgcctaatcgtcaaggaagaaggtgtaatcagtgtcaaccaggtttttatatttctccaggcaatgccactggctgcctgccatgctcatgccatacaactggtgcagttaatcacatctgtaatagcctgactggtcagtgtgtttgccaagatgcttccattgctgggcaacgttgtgaccaatgcaaagaccattactttggatttgatcctcagactggaagatgtcagccttgtaattgtcatctctcaggagccttgaatgaaacctgtcacttggtcacaggccagtgtttctgtaaacaatttgtcactggctcaaagtgtgatgcttgtgttcccagtgcaagccacttggatgtcaacaatctattgggttgcagcaaaactccattccagcaacctccgcccagaggacaagttcaaagttcttctgctatcaatctctcctggagtccacctgattctccaaatgcccactggcttacttacagtttactcagggatggttttgaaatctacacaacagaggatcaatacccatacagtattcaatacttcttagacacagacctgttaccatataccaaatattcctattacattgagaccaccaatgtgcatggttcaacaaggagtgtagctgtcacttacaagacaaaaccaggggtcccagagggaaacttgactttaagttatatcattcctattggctcagactctgtgacacttacctggacaacactctcaaatcaatctggtcccatagagaaatatattttgtcctgtgcccctttggctggtggtcagccatgtgtttcctacgaaggtcatgaaacctcagctaccatctggaatctggttccatttgccaagtacgatttttctgtacaggcgtgtactagcgggggctgtttacacagcttgcccattacagtgaccacagcccaggcccctccccaaagactaagtccacctaagatgcagaaaatcagttctacagaacttcatgtagaatggtctccaccagcggaactaaatggaataattataagatatgaactatacatgagaagactgagatctactaaagaaaccacatctgaggaaagtcgagtttttcagagcagtggttggctcagtcctcattcatttgtagaatcggccaatgaaaatgcattaaaacctcctcaaacaatgacaaccatcactggcttggagccatacaccaagtatgagttcagagtcttagctgtgaatatggctggaagtgtgtcttctgcctgggtctcagaaagaacgggagaatcagcacctgtattcatgatccctccttcagtctttcccctctcttcgtactctctcaatatctcctgggagaagccagcagataatgttacaagaggaaaagttgtggggtatgacatcaatatgctttctgaacaatcacctcaacagtctattcccatggcgttttcacagctgttgcacactgctaaatcccaagaactatcttacactgtagaaggactgaaaccttataggatatatgagtttactattactctctgcaattcagttggttgtgtgaccagtgcttcgggagcaggacaaactttagcagcagcaccagcacaactgaggccacctctggttaaaggaatcaacagcacaacaatccatcttaggtggtttccacctgaagaactgaatggaccctctcctatatatcagctggaaaggagagagtcatctctaccagctctgatgaccacgatgatgaaaggaatccgtttcataggaaatgggtattgtaaatttcccagctccactcacccagtcaatacagacttcactggcattaaggccagctttcgaacaaaagtgcctgaaggtttgattgtctttgcagcatcacctggcaatcaggaagagtattttgcacttcagttgaagaagggacgtctttattttctttttgatcctcaggggtcaccagtggaagtaactacaactaatgatcatggcaaacaatatagtgatggaaaatggcatgaaataattgctattaggcatcaggcttttggccaaatcactctggatgggatatatacaggttcctctgccatcctgaatggtagtactgttattggagataacacaggagtctttctgggagggctcccgcgaagttataccatcctcaggaaggatcctgagataatccaaaaaggttttgtgggctgtctcaaggatgtacattttatgaagaattacaatccgtcagctatttgggaacctctggattggcagagttctgaagaacaaatcaacgtgtataacagctgggagggatgtcccgcttcattaaatgagggagctcagttcctaggagcagggttcctggaacttcatccatatatgtttcatggtggaatgaactttgagatttcctttaagttcagaactgaccaattaaatggattgcttcttttcgtttataacaaagatggacctgattttcttgctatggagctgaaaagtggaatattgaccttccggttaaataccagtcttgcctttacacaagtggatctattgctggggctatcctattgtaatggaaagtggaataaagtcattattaaaaaggaaggctctttcatatcagcaagtgtgaatggactgatgaagcatgcatcggagtccggagaccagccactggtggtgaattcaccagtttatgtgggaggaatcccacaggaactgctgaactcttatcaacatttgtgtttggaacaaggtttcggtggttgcatgaaggatgttaaatttacacggggtgctgtcgttaacttggcatctgtgtccagcggtgctgtcagagtcaatctggatggatgcctatcaactgacagtgctgttaactgcaggggaaatgactccatcctggtttaccagggaaaagagcagagtgtttacgagggtggtctccagccttttacagaatacctgtatcgagtgatagcctcgcatgaaggaggttcagtatatagtgattggagtcgaggacgtacaacaggagcagctccacaaagtgtgccaactccctcaagagtccgcagcttaaatggatacagcattgaggtgacctgggatgaacctgttgtcagaggtgtaattgagaagtacattctgaaagcctatagtgaggacagcacccgtccaccccgcatgccctctgccagtgctgaatttgtcaatacaagcaacctcacaggcatattgacaggcttgctacccttcaaaaactatgcagtaaccctaactgcttgcactttggctggctgtactgagagctcacatgcattgaacatctctactccacaagaagccccacaagaggttcagccaccagtagccaaatcccttcccagttctttgctgctctcctggaacccacccaaaaaggcaaatggtattataactcagtactgtttatacatggatgggaggctgatctattcaggcagtgaggagaactacatagtcacagatttagcagtatttacaccccaccagtttctactaagtgcatgcacacatgtgggctgtacaaacagttcctgggtcctactgtacacagcacagctgccaccagaacacgtggattccccagttctgactgtcctggattctagaactatacacatacagtggaaacaaccaagaaaaataagtgggattctggaacgctatgtattatatatgtcaaaccatacacatgattttacaatttggagtgtcatctataacagtacagaacttttccaggatcatatgctacaatacgttttacctggtaataaatatctcatcaagctgggagcttgcacaggtggtgggtgcacagtgagtgaggccagtgaggccctaactgacgaggacatacccgaaggcgtgccagcccccaaagcccactcatattcacctgactcctttaatgtctcctggactgagcctgaatatccgaatggtgttatcacgagttatggattatatctagatggtatattaatccacaattcctcagaactcagctatcgtgcttacggatttgctccttggagtttacattccttcagagtccaagcatgcacggccaaaggttgtgctctgggcccactggtggaaaatcgaactctagaagctcctcctgaaggaacagtaaatgtgtttgtcaaaacacagggatcccggaaagcccacgtgaggtgggaagcaccttttcgccctaatggactcttaacacactcagtccttttcactgggatattctatgtagacccagtaggtaataactacacccttctgaatgtcacaaaagtcatgtacagcggagaagagacaaacctttgggtgctcatcgatgggctggttccttttaccaactatactgtacaagtgaatatttcaaatagccaaggcagcttgataactgatcctataacaattgcaatgcctccaggagctccagatggcgtgctgcctcccaggctttcatctgccactccaaccagtcttcaggttgtctggtctacaccagctcgtaataacgctcctggctctcccagataccaactccagatgaggtctggcgactccacccatggatttctagagttattttccaatccttctgcatcgttaagctatgaagtgagtgatctccaaccgtacacagagtatatgtttcggttggttgcctccaatggatttggcagtgcacatagttcttggattccattcatgaccgcagaggacaaacctggacctgtagttcctccgattcttctggatgtgaagtcaagaatgatgttggtcacctggcagcatcctagaaaatccaatggggttattacccattataacatttatctacatggccgtctatacttgagaactcctggaaatgtcactaattgcacagtgatgcatttacacccatacactgcctataagtttcaggtagaagcctgcacttcaaaaggatgttccctttcaccagagtcccagactgtatggacactcccaggggcaccggaagggatcccaagtccagagctgttctctgatactccaacatctgtgattatatcttggcaaccccctacccaccccaatggcttggtggagaatttcacaattgagagaagagtcaaaggaaaggaagaagttactaccctggtgactctcccgaggagtcattccatgaggtttattgacaagacttctgctcttagcccatggacaaaatatgaatatcgggtactgatgagcactcttcatggaggcacaaacagcagtgcttgggtagaagttaccacaagaccctcacgacctgctggggtgcagccacctgtggtgacagtgctggaacccgatgcagtccaggtcacttggaaacccccactcatccagaacggagacatacttagctatgagattcacatgcctgaccctcacatcactttaaccaatgtgacttccgcagtgttaagtcaaaaagttactcatctgattcctttcactaattattctgtcaccattgttgcttgctcagggggtaatgggtaccttggagggtgcacagagagtttacctacctatgttaccactcaccccaccgtacctcagaatgttggcccattgtctgtgattccactaagtgaatcatatgttgtgatttcttggcaaccaccatccaagccaaatggacctaatttgagatatgagcttctgagacgtaaaatccagcagccacttgcatcaaatcccccagaagatttaaatcggtggcacaatatttattcaggaactcagtggctttatgaagataagggtcttagcaggtttacaacctatgaatatatgctcttcgtacacaacagtgtgggttttacaccgagccgagaagtgactgtgacaacgttagctggtcttccagagagaggagccaatctcactgcgagtgtccttaaccacacagccatcgacgtgaggtgggctaaaccaactgttcaagacctacaaggtgaagttgaatattacacacttttttggagttctgctacctcaaacgactctctaaaaatcttgccagatgtaaactctcatgtcattggccacctaaagccaaacacagagtattggatctttatctctgtcttcaatggagtccacagcatcaacagtgcaggacttcatgcaaccacttgcgatggggagcctcagggcatgcttcctccagaggttgtcatcatcaacagtacagctgtacgtgtcatctggacatctccttcaaacccaaatggtgttgtcactgagtattctatctatgtaaataataagctctacaagactggaatgaatgtgcctgggtcgtttattctgagagacctgtctcccttcactatctatgacattcaggttgaagtctgcacaatatatgcctgcgtgaaaagcaatggaacccaaattaccactgtggaagacactccaagtgatataccaacacccacaattcgtggcatcacttcaagatctcttcaaattgattgggtgtctccacggaagccaaatggcatcattcttggatatgatctcctatggaaaacatggtatccatgcgctaaaactcaaaagttagtgcaggatcagagtgatgagctctgcaaggcagtgaggtgtcaaaaacctgaatctatctgtggacacatttgctattcttctgaagctaaggtttgttgtaacggagtgctctataaccccaagcctggacatcgctgttgtgaagaaaagtatatcccgtttgttctgaattctactggagtttgttgtggtggccgaatacaggaggcacaaccaaatcatcagtgctgctctgggtattacgctagaattctaccaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtatgttctgttgtgggcaggattatgtgaatatgtcagataccatatgctgctcagcttccagtggagagtctaaagcacatattaaaaagaatgacccggtgccagtaaaatgctgtgagactgaacttattccaaagagccagaaatgctgtaatggagttggatataatcctttgaaatatgtttgctctgacaagatttcaactggaatgatgatgaaggaaaccaaagagtgcaggatcctctgcccagcatctatggaagccacagaacattgtggcaggtgtgacttcaactttaccagccacatttgcactgtgataagagggtctcacaattccacagggaaggcatcaattgaagaaatgtgttcatctgccgaagaaaccattcatacagggagtgtaaacacgtactcttacacagatgtgaacctcaagccctacatgacatatgagtacaggatttctgcctggaacagctatgggcgaggactcagcaaagctgtgagagccagaacaaaagaagatgtgcctcaaggagtgagtccccctacgtggaccaaaatagacaatcttgaagatacaattgtcttaaactggagaaaacctatacaatcaaatggtcctattatttactacatccttcttcgaaatggaattgaacgttttcggggaacatcactgagcttctctgataaagagggaattcaaccatttcaggaatattcatatcagctgaaagcttgcacggttgctggctgtgccaccagtagcaaggtagttgcagctactacccaaggagttccggagagcatcctgccaccaagcatcacagccctaagtgcagtggctctgcatctgagctggagtgtccctgagaaatcaaacggcgtcattaaagagtaccagatcaggcaggttgggaaaggtctcatccacactgacaccactgacaggagacagcatacggtcacaggtctccagccatacaccaactacagcttcactcttacagcttgtacatctgctgggtgcacttcaagcgagccttttctaggtcagacactgcaggcagctcctgaaggagtttgggtgacacctcgacacattatcatcaattctacaacagtggaattatattggagtctgccagaaaagcccaatggcctcgtttctcaatatcaattgagtcgtaatggaaacttgcttttcctgggtggcagtgaggagcagaatttcactgataaaaacctggagcccaatagcagatacacttacaagttagaagtcaaaactggaggtggcagcagtgctagtgatgattacattgttcaaacacctatgtcaacaccagaagaaatctatcctccatataatatcacagtaattgggccttattctatatttgtagcttggataccaccagggatcctcatccccgaaattcctgtggagtacaatgtcttactcaatgatggaagtgtaacacctctggccttctccgttggtcatcatcaatccacccttctggaaaatttgactccattcacacagtatgagataaggatacaagcatgtcaaaatggaagttgtggagttagcagtaggatgtttgtcaaaacacctgaagcagccccaatggatcttaattctcctgttcttaaggcactggggtcagcttgcatagagattaagtggatgccacctgaaaaaccaaatggaatcatcatcaactactttatttacagacgccctgctggcattgaagaggagtctgttttatttgtctggtcagaaggagcccttgaatttatggatgaaggagacaccctgaggcctttcacactctacgaatatcgggtcagagcctgtaactccaagggttcagtggagagtctgtggtcattaacacaaactctggaagctccacctcaagattttccagctccttgggctcaagccacgagtgctcattcagttctgttgaattggacaaagccagaatctcccaatggcattatctcccattaccgtgtggtctaccaggagagacccgacgatcctacatttaacagccctaccgtgcatgctttcacagtgaagggaacaagccatcaagcccacctgtacgggttagaaccattcacaacatatcgcattggtgttgtggctgcaaaccatgcaggagaaattttaagcccttggactctgattcaaaccttagaatcttccccaagtggactgagaaactttatagtagaacagaaagagaatggccgggcattgctactacagtggtcagaacctatgagaaccaatggtgtgattaagacatacaacatcttcagtgacgggttcctggagtactctggtttgaatcgtcagtttctcttccgccgcctggatcctttcactctctacacactgaccctggaggcctgcaccagagcaggttgtgcacactcggcgcctcagcctctgtggacagatgaagcccctccagactctcagctggctcctactgtccactctgtgaagtccaccagtgttgagctgagctggtctgagcctgttaacccaaatggaaaaataattcgctatgaagtgattcgcagatgcttcgagggaaaagcttggggaaatcagacaatccaggccgacgagaaaattgttttcacagaatataacactgaaaggaatacatttatgtataatgacacaggtttgcaaccatggacgcagtgtgaatataaaatctacacttggaattcagctgggcatacctgtagctcttggaatgtggtgaggacattgcaagcacctccagaaggtctctctccacctgtgatatcctatgtttctatgaatccccaaaaactgctgatttcctggatcccaccagaacagtctaatggtattatccagtcctataggcttcaaaggaatgaaatgctctatccttttagctttgatcctgtgactttcaattacactgatgaagagcttcttcctttttccacctatagctatgcactccaagcctgcacgagtggaggatgctccaccagcaaacccaccagcatcacaactctggaggctgctccatcagaagtcagccctccagatctttgggccgtcagtgccactcaaatgaatgtatgttggtcaccgcccacagtgcaaaatggaaagattactaaatatttagttagatatgataataaagagtcccttgctggccagggcctgtgcctgctggtttcccacctgcagccttactctcagtataacttctcccttgtagcctgcacgaatggaggttgcacagctagtgtgtcaaaatctgcctggacaatggaggccctgccagagaacatggactctccaacattgcaagtcacaggctcagaatcaatagaaatcacctggaaacctccaagaaacccaaatggccagatcagaagttatgaacttaggagggatggaaccattgtatatacaggcttggaaacacgctatcgtgattttactctcaccccaggtgtggagtatagctacacagtaactgccagcaacagccaagggggtattttgagtcctcttgtcaaagatcgaaccagcccctcagcaccctcagggatggaacctccaaaattgcaggccaggggtcctcaggagatcttagtgaactgggaccctccagtgagaacaaatggtgatatcatcaattataccctcttcatccgtgaactatttgaaagagaaactaaaatcatacacataaacacaactcataattcttttggtatgcagtcatatatagtaaaccagctgaagccatttcacaggtatgaaatacgaattcaagcgtgcaccaccctgggatgtgcatcaagtgactggacattcatacagacccctgagattgcacctttgatgcaaccccctccacatctggaggtacaaatggctccaggaggattccagccaactgtttctcttttgtggacaggaccgctgcagccaaatggaaaagttttgtattacgaattatacagaagacaaatagcaactcagcctagaaaatccaatccagtcctaatctataacggaagctcaacatcttttatagattccgaactattgcctttcacagagtatgagtatcaggtctgggcagtgaattctgcaggaaaagcccccagtagctggacatggtgcagaaccgggccagccccaccagaaggtctcagagcccccacgttccatgtgatctcttctacccaagcagtggtcaacatcagtgcccctgggaagcccaacgggatcgtcagtctctacaggctgttctccagcagcgcccatggggctgagacagtgctatccgaaggcatggccacccagcagactctccatggccttcaagccttcactaactactctattggagtagaggcctgcacctgcttcaactgttgcagcaaaggaccgacagctgaactgagaacccatcctgccccaccctcaggactgtcctctccacaaatcgggacgctggcctcaaggacggcctccttccggtggagtccccccatgttccccaatggtgtcattcacagctatgaactccaattccacgtggcttgccctcctgactcagccctcccctgtactcccagccaaatagaaacaaagtacacggggctggggcagaaagccagccttgggggtctccagccctacaccacatacaagctgagagtggtggcacacaacgaggtgggcagtacggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctg

SEQ ID NO: 3 - protein, 40 aa, Artificial Sequence
  polypeptide fragment
MNCPVLSLGSGFLFQVIEMLIFAYFASISLTESRGLFPRL

SEQ ID NO: 4 - DNA, 120 bp, Artificial Sequence
  polynucleotide fragment
atgaattgcccagttctttcattgggctctggcttcttgtttcaggtcattgaaatgttgatctttgcctattttgcttcaatatccttgactgagtcacgaggtcttttcccaaggctg

SEQ ID NO: 5 - protein, 23 aa, Artificial Sequence
  polypeptide fragment
LWFIVLMAMLGLILLAIFLSLIL

SEQ ID NO: 6 - DNA, 69 bp, Artificial Sequence
  polynucleotide fragment
ctgtggttcatagtgttaatggcgatgctgggcttgatcttgttggccatttttctgtccctgatacta

SEQ ID NO: 7 - protein, 139 aa, Artificial Sequence
  polypeptide fragment
QRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 8 - DNA, 417 bp, Artificial Sequence
  polynucleotide fragment
caaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctg

SEQ ID NO: 9 - protein, 83 aa, Artificial Sequence
  polypeptide fragment
PERGANLTASVLNHTAIDVRWAKPTVQDLQGEVEYYTLFWSSATSNDSLKILPDVNSHVIGHLKPNTEYWIFISVFNGVHSIN

SEQ ID NO: 10 - DNA, 249 bp, Artificial Sequence
  polynucleotide fragment
ccagagagaggagccaatctcactgcgagtgtccttaaccacacagccatcgacgtgaggtgggctaaaccaactgttcaagacctacaaggtgaagttgaatattacacacttttttggagttctgctacctcaaacgactctctaaaaatcttgccagatgtaaactctcatgtcattggccacctaaagccaaacacagagtattggatctttatctctgtcttcaatggagtccacagcatcaac

SEQ ID NO: 11 - protein, 77 aa, Artificial Sequence
  polypeptide fragment
PQGMLPPEVVIINSTAVRVIWTSPSNPNGVVTEYSIYVNNKLYKTGMNVPGSFILRDLSPFTIYDIQVEVCTIYACV

SEQ ID NO: 12 - DNA, 231 bp, Artificial Sequence
  polynucleotide fragment
cctcagggcatgcttcctccagaggttgtcatcatcaacagtacagctgtacgtgtcatctggacatctccttcaaacccaaatggtgttgtcactgagtattctatctatgtaaataataagctctacaagactggaatgaatgtgcctgggtcgtttattctgagagacctgtctcccttcactatctatgacattcaggttgaagtctgcacaatatatgcctgcgtg

SEQ ID NO: 13 - protein, 75 aa, Artificial Sequence
  polypeptide fragment
VSPPTWTKIDNLEDTIVLNWRKPIQSNGPIIYYILLRNGIERFRGTSLSFSDKEGIQPFQEYSYQLKACTVAGCA

SEQ ID NO: 14 - DNA, 225 bp, Artificial Sequence
  polynucleotide fragment
gtgagtccccctacgtggaccaaaatagacaatcttgaagatacaattgtcttaaactggagaaaacctatacaatcaaatggtcctattatttactacatccttcttcgaaatggaattgaacgttttcggggaacatcactgagcttctctgataaagagggaattcaaccatttcaggaatattcatatcagctgaaagcttgcacggttgctggctgtgcc

SEQ ID NO: 15 - protein, 78 aa, Artificial Sequence
  polypeptide fragment
PESILPPSITALSAVALHLSWSVPEKSNGVIKEYQIRQVGKGLIHTDTTDRRQHTVTGLQPYTNYSFTLTACTSAGCT

SEQ ID NO: 16 - DNA, 234 bp, Artificial Sequence
  polynucleotide fragment
ccggagagcatcctgccaccaagcatcacagccctaagtgcagtggctctgcatctgagctggagtgtccctgagaaatcaaacggcgtcattaaagagtaccagatcaggcaggttgggaaaggtctcatccacactgacaccactgacaggagacagcatacggtcacaggtctccagccatacaccaactacagcttcactcttacagcttgtacatctgctgggtgcact

SEQ ID NO: 17 - protein, 78 aa, Artificial Sequence
  polypeptide fragment
PEGVWVTPRHIIINSTTVELYWSLPEKPNGLVSQYQLSRNGNLLFLGGSEEQNFTDKNLEPNSRYTYKLEVKTGGGSS

SEQ ID NO: 18 - DNA, 234 bp, Artificial Sequence
  polynucleotide fragment
cctgaaggagtttgggtgacacctcgacacattatcatcaattctacaacagtggaattatattggagtctgccagaaaagcccaatggcctcgtttctcaatatcaattgagtcgtaatggaaacttgcttttcctgggtggcagtgaggagcagaatttcactgataaaaacctggagcccaatagcagatacacttacaagttagaagtcaaaactggaggtggcagcagt

SEQ ID NO: 19 - protein, 84 aa, Artificial Sequence
  polypeptide fragment
PEEIYPPYNITVIGPYSIFVAWIPPGILIPEIPVEYNVLLNDGSVTPLAFSVGHHQSTLLENLTPFTQYEIRIQACQNGSCGVS

SEQ ID NO: 20 - DNA, 252 bp, Artificial Sequence
  polynucleotide fragment
ccagaagaaatctatcctccatataatatcacagtaattgggccttattctatatttgtagcttggataccaccagggatcctcatccccgaaattcctgtggagtacaatgtcttactcaatgatggaagtgtaacacctctggccttctccgttggtcatcatcaatccacccttctggaaaatttgactccattcacacagtatgagataaggatacaagcatgtcaaaatggaagttgtggagttagc

SEQ ID NO: 21 - protein, 88 aa, Artificial Sequence
  polypeptide fragment
EAAPMDLNSPVLKALGSACIEIKWMPPEKPNGIIINYFIYRRPAGIEEESVLFVWSEGALEFMDEGDTLRPFTLYEYRVRACNSKGSV

SEQ ID NO: 22 - DNA, 264 bp, Artificial Sequence
  polynucleotide fragment
gaagcagccccaatggatcttaattctcctgttcttaaggcactggggtcagcttgcatagagattaagtggatgccacctgaaaaaccaaatggaatcatcatcaactactttatttacagacgccctgctggcattgaagaggagtctgttttatttgtctggtcagaaggagcccttgaatttatggatgaaggagacaccctgaggcctttcacactctacgaatatcgggtcagagcctgtaactccaagggttcagtg

SEQ ID NO: 23 - protein, 376 aa, Artificial Sequence
  polypeptide fragment
PSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRG

SEQ ID NO: 24 - DNA, 1128 bp, Artificial Sequence
  polynucleotide fragment
ccaagtgatataccaacacccacaattcgtggcatcacttcaagatctcttcaaattgattgggtgtctccacggaagccaaatggcatcattcttggatatgatctcctatggaaaacatggtatccatgcgctaaaactcaaaagttagtgcaggatcagagtgatgagctctgcaaggcagtgaggtgtcaaaaacctgaatctatctgtggacacatttgctattcttctgaagctaaggtttgttgtaacggagtgctctataaccccaagcctggacatcgctgttgtgaagaaaagtatatcccgtttgttctgaattctactggagtttgttgtggtggccgaatacaggaggcacaaccaaatcatcagtgctgctctgggtattacgctagaattctaccaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtatgttctgttgtgggcaggattatgtgaatatgtcagataccatatgctgctcagcttccagtggagagtctaaagcacatattaaaaagaatgacccggtgccagtaaaatgctgtgagactgaacttattccaaagagccagaaatgctgtaatggagttggatataatcctttgaaatatgtttgctctgacaagatttcaactggaatgatgatgaaggaaaccaaagagtgcaggatcctctgcccagcatctatggaagccacagaacattgtggcaggtgtgacttcaactttaccagccacatttgcactgtgataagagggtctcacaattccacagggaaggcatcaattgaagaaatgtgttcatctgccgaagaaaccattcatacagggagtgtaaacacgtactcttacacagatgtgaacctcaagccctacatgacatatgagtacaggatttctgcctggaacagctatgggcgagga

SEQ ID NO: 25 - protein, 138 aa, Artificial Sequence
  polypeptide fragment
ASFTLAVWLKPEQQGVMCVIEKTVDGQIVFKLTISEKETMFYYRTVNGLQPPIKVMTLGRILVKKWIHLSVQVHQTKISFFINGVEKDHTPFNARTLSGSITDFASGTVQIGQSLNGLEQFVGRMQDFRLYQVALTNR

SEQ ID NO: 26 - DNA, 414 bp, Artificial Sequence
  polynucleotide fragment
gcatcatttaccttagctgtatggctgaaacctgagcaacaaggtgtaatgtgtgttatagaaaagacagtagatgggcagattgtgttcaaacttacaatatctgagaaagagaccatgttttattatcgcacagtaaatggtttgcaacctccaataaaagtaatgacactggggagaattcttgtgaagaaatggattcatcttagtgtgcaggtgcatcagacaaaaatcagcttctttatcaatggcgtggagaaggatcatacacctttcaatgcaagaactctaagtggttcaattacagattttgcatctggtactgtgcaaataggacagagtttaaatggtttagagcagtttgtcggaagaatgcaagattttcgattataccaagtggcacttacaaacaga

SEQ ID NO: 27 - protein, 243 aa, Artificial Sequence
  polypeptide fragment
RLYQVALTNREILEVFSGDLLRLHAQSHCRCPGSHPRVHPLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQEFVKATQIRFHFHGQYYTTETAVNLRHRYYAVDEITISG

SEQ ID NO: 28 - DNA, 729 bp, Artificial Sequence
  polynucleotide fragment
cgattataccaagtggcacttacaaacagagagattctggaagtcttctctggagatcttctcagattgcatgcccaatcacattgccgttgccctggcagccacccgcgggtccaccctttggcacagcggtactgcattcctaatgatgcaggagacacagctgataatagagtgtcacggttgaatcctgaagcccatcctctctcttttgtcaatgataatgatgttggtacttcatgggtttcaaatgtgtttacaaacattacacagcttaatcaaggagtgactatttcagttgatttggaaaatggacagtatcaggtgttttatattatcattcagttctttagtccacaaccaacggaaataaggattcaaaggaagaaggaaaatagtttagattgggaggactggcaatattttgccaggaattgtggtgcttttggaatgaaaaacaatggagatttggaaaaacctgattctgtcaactgtcttcagctttccaattttactccatattcccgtggcaatgtcacatttagcatcctgacacctggaccaaattatcgtcctggatacaataacttctataataccccatctcttcaagagttcgtaaaagccacgcaaataaggtttcattttcatgggcagtactatacaactgagactgctgttaacctcagacacagatattatgcagtggacgaaatcaccattagtggg

SEQ ID NO: 29 - protein, 55 aa, Artificial Sequence
  polypeptide fragment
CQCHGHADNCDTTSQPYRCLCSQESFTEGLHCDRCLPLYNDKPFRQGDQVYAFNC

SEQ ID NO: 30 - DNA, 165 bp, Artificial Sequence
  polynucleotide fragment
tgtcagtgccatggtcatgccgataactgcgacacaacaagccagccatatagatgcctctgctcccaggagagcttcactgaaggacttcattgtgatcgctgcttgcctctttataatgacaagcctttccgccaaggtgatcaagtttacgctttcaattgt

SEQ ID NO: 31 - protein, 128 aa, Artificial Sequence
  polypeptide fragment
CQCNSHSKSCHYNISVDPFPFEHFRGGGGVCDDCEHNTTGRNCELCKDYFFRQVGADPSAIDVCCQCNSHSKSCHYNISVDPFPFEHFRGGGGVCDDCEHNTTGRNCELCKDYFFRQVGADPSAIDVC

SEQ ID NO: 32 - DNA, 192 bp, Artificial Sequence
  polynucleotide fragment
tgtcaatgcaacagccattccaaaagctgccattacaacatctctgtagacccatttccttttgagcacttcagagggggaggaggagtttgtgatgattgtgagcataacactacaggaaggaactgtgagctgtgcaaggattactttttccgacaagttggtgcagatccttcggccatagatgtttgc

SEQ ID NO: 33 - protein, 51 aa, Artificial Sequence
  polypeptide fragment
CDCDTVGTRNGSILCDQIGGQCNCKRHVSGRQCNQCQNGFYNLQELDPDGC

SEQ ID NO: 34 - DNA, 153 bp, Artificial Sequence
  polynucleotide fragment
tgtgactgtgatacagttggcactagaaatggtagcattctttgtgatcagattggaggacagtgtaattgtaagagacacgtgtctggcaggcagtgcaatcagtgccagaatggattctacaatctacaagagttggatcctgatggctgc

SEQ ID NO: 35 - protein, 51 aa, Artificial Sequence
  polypeptide fragment
CNCNTSGTVDGDITCHQNSGQCKCKANVIGLRCDHCNFGFKFLRSFNDVGC

SEQ ID NO: 36 - DNA, 153 bp, Artificial Sequence
  polynucleotide fragment
tgtaactgcaatacctctgggacagtggatggagatattacctgtcaccaaaattcaggccagtgcaagtgcaaagcaaacgttattgggcttaggtgtgatcattgcaattttggatttaaatttctccgaagctttaatgatgttggatgt

SEQ ID NO: 37 - protein, 136 aa, Artificial Sequence
  polypeptide fragment
MNFEISFKFRTDQLNGLLLFVYNKDGPDFLAMELKSGILTFRLNTSLAFTQVDLLLGLSYCNGKWNKVIIKKEGSFISASVNGLMKHASESGDQPLVVNSPVYVGGIPQELLNSYQHLCLEQGFGGCMKDVKFTRG

SEQ ID NO: 38 - DNA, 408 bp, Artificial Sequence
  polynucleotide fragment
atgaactttgagatttcctttaagttcagaactgaccaattaaatggattgcttcttttcgtttataacaaagatggacctgattttcttgctatggagctgaaaagtggaatattgaccttccggttaaataccagtcttgcctttacacaagtggatctattgctggggctatcctattgtaatggaaagtggaataaagtcattattaaaaaggaaggctctttcatatcagcaagtgtgaatggactgatgaagcatgcatcggagtccggagaccagccactggtggtgaattcaccagtttatgtgggaggaatcccacaggaactgctgaactcttatcaacatttgtgtttggaacaaggtttcggtggttgcatgaaggatgttaaatttacacggggt

SEQ ID NO: 39 - protein, 2262 aa, Artificial Sequence
  MiniUSH2A-1
MNCPVLSLGSGFLFQVIEMLIFAYFASISLTESRGLFPRLENVGAFKKVSIVPTQAVCGLPDRSTFCHSSAAAESIQFCTQRFCIQDCPYRSSHPTYTALFSAGLSSCITPDKNDLHPNAHSNSASFIFGNHKSCFSSPPSPKLMASFTLAVWLKPEQQGVMCVIEKTVDGQIVFKLTISEKETMFYYRTVNGLQPPIKVMTLGRILVKKWIHLSVQVHQTKISFFINGVEKDHTPFNARTLSGSITDFASGTVQIGQSLNGLEQFVGRMQDFRLYQVALTNREILEVFSGDLLRLHAQSHCRCPGSHPRVHPLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQEFVKATQIRFHFHGQYYTTETAVNLRHRYYAVDEITISGRCQCHGHADNCDTTSQPYRCLCSQESFTEGLHCDRCLPLYNDKPFRQGDQVYAFNCKPCQCNSHSKSCHYNISVDPFPFEHFRGGGGVCDDCEHNTTGRNCELCKDYFFRQVGADPSAIDVCKPCDCDTVGTRNGSILCDQIGGQCNCKRHVSGRQCNQCQNGFYNLQELDPDGCSPCNCNTSGTVDGDITCHQNSGQCKCKANVIGLRCDHCNFGFKFLRSFNDVGCYNPSAIWEPLDWQSSEEQINVYNSWEGCPASLNEGAQFLGAGFLELHPYMFHGGMNFEISFKFRTDQLNGLLLFVYNKDGPDFLAMELKSGILTFRLNTSLAFTQVDLLLGLSYCNGKWNKVIIKKEGSFISASVNGLMKHASESGDQPLVVNSPVYVGGIPQELLNSYQHLCLEQGFGGCMKDVKFTRGPSREVTVTTLAGLPERGANLTASVLNHTAIDVRWAKPTVQDLQGEVEYYTLFWSSATSNDSLKILPDVNSHVIGHLKPNTEYWIFISVFNGVHSINSAGLHATTCDGEPQGMLPPEVVIINSTAVRVIWTSPSNPNGVVTEYSIYVNNKLYKTGMNVPGSFILRDLSPFTIYDIQVEVCTIYACVKSNGTQITTVEDTPSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRGLSKAVRARTKEDVPQGVSPPTWTKIDNLEDTIVLNWRKPIQSNGPIIYYILLRNGIERFRGTSLSFSDKEGIQPFQEYSYQLKACTVAGCATSSKVVAATTQGVPESILPPSITALSAVALHLSWSVPEKSNGVIKEYQIRQVGKGLIHTDTTDRRQHTVTGLQPYTNYSFTLTACTSAGCTSSEPFLGQTLQAAPEGVWVTPRHIIINSTTVELYWSLPEKPNGLVSQYQLSRNGNLLFLGGSEEQNFTDKNLEPNSRYTYKLEVKTGGGSSASDDYIVQTPMSTPEEIYPPYNITVIGPYSIFVAWIPPGILIPEIPVEYNVLLNDGSVTPLAFSVGHHQSTLLENLTPFTQYEIRIQACQNGSCGVSSRMFVKTPEAAPMDLNSPVLKALGSACIEIKWMPPEKPNGIIINYFIYRRPAGIEEESVLFVWSEGALEFMDEGDTLRPFTLYEYRVRACNSKGSVESLWASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 40 - DNA, 6786 bp, Artificial Sequence
  MiniUSH2A-1
atgaattgcccagttctttcattgggctctggcttcttgtttcaggtcattgaaatgttgatctttgcctattttgcttcaatatccttgactgagtcacgaggtcttttcccaaggctggagaacgtgggagctttcaagaaagtttccatcgtgccaacccaagcagtatgtggactcccagaccgaagcactttttgtcacagctctgctgctgctgaaagtattcagttctgtacccagcggttttgtattcaggattgcccatacagatcttcacaccctacctacactgcccttttctcagcaggcctcagtagctgcatcacaccagacaagaatgatctgcatcctaacgcccatagcaattctgcaagttttatttttggaaatcacaagagctgcttttcttctcctccttctccaaagctgatggcatcatttaccttagctgtatggctgaaacctgagcaacaaggtgtaatgtgtgttatagaaaagacagtagatgggcagattgtgttcaaacttacaatatctgagaaagagaccatgttttattatcgcacagtaaatggtttgcaacctccaataaaagtaatgacactggggagaattcttgtgaagaaatggattcatcttagtgtgcaggtgcatcagacaaaaatcagcttctttatcaatggcgtggagaaggatcatacacctttcaatgcaagaactctaagtggttcaattacagattttgcatctggtactgtgcaaataggacagagtttaaatggtttagagcagtttgtcggaagaatgcaagattttcgattataccaagtggcacttacaaacagagagattctggaagtcttctctggagatcttctcagattgcatgcccaatcacattgccgttgccctggcagccacccgcgggtccaccctttggcacagcggtactgcattcctaatgatgcaggagacacagctgataatagagtgtcacggttgaatcctgaagcccatcctctctcttttgtcaatgataatgatgttggtacttcatgggtttcaaatgtgtttacaaacattacacagcttaatcaaggagtgactatttcagttgatttggaaaatggacagtatcaggtgttttatattatcattcagttctttagtccacaaccaacggaaataaggattcaaaggaagaaggaaaatagtttagattgggaggactggcaatattttgccaggaattgtggtgcttttggaatgaaaaacaatggagatttggaaaaacctgattctgtcaactgtcttcagctttccaattttactccatattcccgtggcaatgtcacatttagcatcctgacacctggaccaaattatcgtcctggatacaataacttctataataccccatctcttcaagagttcgtaaaagccacgcaaataaggtttcattttcatgggcagtactatacaactgagactgctgttaacctcagacacagatattatgcagtggacgaaatcaccattagtgggagatgtcagtgccatggtcatgccgataactgcgacacaacaagccagccatatagatgcctctgctcccaggagagcttcactgaaggacttcattgtgatcgctgcttgcctctttataatgacaagcctttccgccaaggtgatcaagtttacgctttcaattgtaaaccttgtcaatgcaacagccattccaaaagctgccattacaacatctctgtagacccatttccttttgagcacttcagagggggaggaggagtttgtgatgattgtgagcataacactacaggaaggaactgtgagctgtgcaaggattactttttccgacaagttggtgcagatccttcggccatagatgtttgcaaaccctgtgactgtgatacagttggcactagaaatggtagcattctttgtgatcagattggaggacagtgtaattgtaagagacacgtgtctggcaggcagtgcaatcagtgccagaatggattctacaatctacaagagttggatcctgatggctgcagtccctgtaactgcaatacctctgggacagtggatggagatattacctgtcaccaaaattcaggccagtgcaagtgcaaagcaaacgttattgggcttaggtgtgatcattgcaattttggatttaaatttctccgaagctttaatgatgttggatgttacaatccgtcagctatttgggaacctctggattggcagagttctgaagaacaaatcaacgtgtataacagctgggagggatgtcccgcttcattaaatgagggagctcagttcctaggagcagggttcctggaacttcatccatatatgtttcatggtggaatgaactttgagatttcctttaagttcagaactgaccaattaaatggattgcttcttttcgtttataacaaagatggacctgattttcttgctatggagctgaaaagtggaatattgaccttccggttaaataccagtcttgcctttacacaagtggatctattgctggggctatcctattgtaatggaaagtggaataaagtcattattaaaaaggaaggctctttcatatcagcaagtgtgaatggactgatgaagcatgcatcggagtccggagaccagccactggtggtgaattcaccagtttatgtgggaggaatcccacaggaactgctgaactcttatcaacatttgtgtttggaacaaggtttcggtggttgcatgaaggatgttaaatttacacggggtccgagccgagaagtgactgtgacaacgttagctggtcttccagagagaggagccaatctcactgcgagtgtccttaaccacacagccatcgacgtgaggtgggctaaaccaactgttcaagacctacaaggtgaagttgaatattacacacttttttggagttctgctacctcaaacgactctctaaaaatcttgccagatgtaaactctcatgtcattggccacctaaagccaaacacagagtattggatctttatctctgtcttcaatggagtccacagcatcaacagtgcaggacttcatgcaaccacttgcgatggggagcctcagggcatgcttcctccagaggttgtcatcatcaacagtacagctgtacgtgtcatctggacatctccttcaaacccaaatggtgttgtcactgagtattctatctatgtaaataataagctctacaagactggaatgaatgtgcctgggtcgtttattctgagagacctgtctcccttcactatctatgacattcaggttgaagtctgcacaatatatgcctgcgtgaaaagcaatggaacccaaattaccactgtggaagacactccaagtgatataccaacacccacaattcgtggcatcacttcaagatctcttcaaattgattgggtgtctccacggaagccaaatggcatcattcttggatatgatctcctatggaaaacatggtatccatgcgctaaaactcaaaagttagtgcaggatcagagtgatgagctctgcaaggcagtgaggtgtcaaaaacctgaatctatctgtggacacatttgctattcttctgaagctaaggtttgttgtaacggagtgctctataaccccaagcctggacatcgctgttgtgaagaaaagtatatcccgtttgttctgaattctactggagtttgttgtggtggccgaatacaggaggcacaaccaaatcatcagtgctgctctgggtattacgctagaattctaccaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtatgttctgttgtgggcaggattatgtgaatatgtcagataccatatgctgctcagcttccagtggagagtctaaagcacatattaaaaagaatgacccggtgccagtaaaatgctgtgagactgaacttattccaaagagccagaaatgctgtaatggagttggatataatcctttgaaatatgtttgctctgacaagatttcaactggaatgatgatgaaggaaaccaaagagtgcaggatcctctgcccagcatctatggaagccacagaacattgtggcaggtgtgacttcaactttaccagccacatttgcactgtgataagagggtctcacaattccacagggaaggcatcaattgaagaaatgtgttcatctgccgaagaaaccattcatacagggagtgtaaacacgtactcttacacagatgtgaacctcaagccctacatgacatatgagtacaggatttctgcctggaacagctatgggcgaggactcagcaaagctgtgagagccagaacaaaagaagatgtgcctcaaggagtgagtccccctacgtggaccaaaatagacaatcttgaagatacaattgtcttaaactggagaaaacctatacaatcaaatggtcctattatttactacatccttcttcgaaatggaattgaacgttttcggggaacatcactgagcttctctgataaagagggaattcaaccatttcaggaatattcatatcagctgaaagcttgcacggttgctggctgtgccaccagtagcaaggtagttgcagctactacccaaggagttccggagagcatcctgccaccaagcatcacagccctaagtgcagtggctctgcatctgagctggagtgtccctgagaaatcaaacggcgtcattaaagagtaccagatcaggcaggttgggaaaggtctcatccacactgacaccactgacaggagacagcatacggtcacaggtctccagccatacaccaactacagcttcactcttacagcttgtacatctgctgggtgcacttcaagcgagccttttctaggtcagacactgcaggcagctcctgaaggagtttgggtgacacctcgacacattatcatcaattctacaacagtggaattatattggagtctgccagaaaagcccaatggcctcgtttctcaatatcaattgagtcgtaatggaaacttgcttttcctgggtggcagtgaggagcagaatttcactgataaaaacctggagcccaatagcagatacacttacaagttagaagtcaaaactggaggtggcagcagtgctagtgatgattacattgttcaaacacctatgtcaacaccagaagaaatctatcctccatataatatcacagtaattgggccttattctatatttgtagcttggataccaccagggatcctcatccccgaaattcctgtggagtacaatgtcttactcaatgatggaagtgtaacacctctggccttctccgttggtcatcatcaatccacccttctggaaaatttgactccattcacacagtatgagataaggatacaagcatgtcaaaatggaagttgtggagttagcagtaggatgtttgtcaaaacacctgaagcagccccaatggatcttaattctcctgttcttaaggcactggggtcagcttgcatagagattaagtggatgccacctgaaaaaccaaatggaatcatcatcaactactttatttacagacgccctgctggcattgaagaggagtctgttttatttgtctggtcagaaggagcccttgaatttatggatgaaggagacaccctgaggcctttcacactctacgaatatcgggtcagagcctgtaactccaagggttcagtggagagtctgtgggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctg

SEQ ID NO: 41 - protein, 1375 aa, Artificial Sequence
  MiniUSH2A-2
MNCPVLSLGSGFLFQVIEMLIFAYFASISLTESRGLFPRLENVGAFKPSREVTVTTLAGLPERGANLTASVLNHTAIDVRWAKPTVQDLQGEVEYYTLFWSSATSNDSLKILPDVNSHVIGHLKPNTEYWIFISVFNGVHSINSAGLHATTCDGEPQGMLPPEVVIINSTAVRVIWTSPSNPNGVVTEYSIYVNNKLYKTGMNVPGSFILRDLSPFTIYDIQVEVCTIYACVKSNGTQITTVEDTPSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRGLSKAVRARTKEDVPQGVSPPTWTKIDNLEDTIVLNWRKPIQSNGPIIYYILLRNGIERFRGTSLSFSDKEGIQPFQEYSYQLKACTVAGCATSSKVVAATTQGVPESILPPSITALSAVALHLSWSVPEKSNGVIKEYQIRQVGKGLIHTDTTDRRQHTVTGLQPYTNYSFTLTACTSAGCTSSEPFLGQTLQAAPEGVWVTPRHIIINSTTVELYWSLPEKPNGLVSQYQLSRNGNLLFLGGSEEQNFTDKNLEPNSRYTYKLEVKTGGGSSASDDYIVQTPMSTPEEIYPPYNITVIGPYSIFVAWIPPGILIPEIPVEYNVLLNDGSVTPLAFSVGHHQSTLLENLTPFTQYEIRIQACQNGSCGVSSRMFVKTPEAAPMDLNSPVLKALGSACIEIKWMPPEKPNGIIINYFIYRRPAGIEEESVLFVWSEGALEFMDEGDTLRPFTLYEYRVRACNSKGSVESLWASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 42 - DNA, 4125 bp, Artificial Sequence
  MiniUSH2A-2
atgaattgcccagttctttcattgggctctggcttcttgtttcaggtcattgaaatgttgatctttgcctattttgcttcaatatccttgactgagtcacgaggtcttttcccaaggctggagaacgtgggagctttcaagccgagccgagaagtgactgtgacaacgttagctggtcttccagagagaggagccaatctcactgcgagtgtccttaaccacacagccatcgacgtgaggtgggctaaaccaactgttcaagacctacaaggtgaagttgaatattacacacttttttggagttctgctacctcaaacgactctctaaaaatcttgccagatgtaaactctcatgtcattggccacctaaagccaaacacagagtattggatctttatctctgtcttcaatggagtccacagcatcaacagtgcaggacttcatgcaaccacttgcgatggggagcctcagggcatgcttcctccagaggttgtcatcatcaacagtacagctgtacgtgtcatctggacatctccttcaaacccaaatggtgttgtcactgagtattctatctatgtaaataataagctctacaagactggaatgaatgtgcctgggtcgtttattctgagagacctgtctcccttcactatctatgacattcaggttgaagtctgcacaatatatgcctgcgtgaaaagcaatggaacccaaattaccactgtggaagacactccaagtgatataccaacacccacaattcgtggcatcacttcaagatctcttcaaattgattgggtgtctccacggaagccaaatggcatcattcttggatatgatctcctatggaaaacatggtatccatgcgctaaaactcaaaagttagtgcaggatcagagtgatgagctctgcaaggcagtgaggtgtcaaaaacctgaatctatctgtggacacatttgctattcttctgaagctaaggtttgttgtaacggagtgctctataaccccaagcctggacatcgctgttgtgaagaaaagtatatcccgtttgttctgaattctactggagtttgttgtggtggccgaatacaggaggcacaaccaaatcatcagtgctgctctgggtattacgctagaattctaccaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtatgttctgttgtgggcaggattatgtgaatatgtcagataccatatgctgctcagcttccagtggagagtctaaagcacatattaaaaagaatgacccggtgccagtaaaatgctgtgagactgaacttattccaaagagccagaaatgctgtaatggagttggatataatcctttgaaatatgtttgctctgacaagatttcaactggaatgatgatgaaggaaaccaaagagtgcaggatcctctgcccagcatctatggaagccacagaacattgtggcaggtgtgacttcaactttaccagccacatttgcactgtgataagagggtctcacaattccacagggaaggcatcaattgaagaaatgtgttcatctgccgaagaaaccattcatacagggagtgtaaacacgtactcttacacagatgtgaacctcaagccctacatgacatatgagtacaggatttctgcctggaacagctatgggcgaggactcagcaaagctgtgagagccagaacaaaagaagatgtgcctcaaggagtgagtccccctacgtggaccaaaatagacaatcttgaagatacaattgtcttaaactggagaaaacctatacaatcaaatggtcctattatttactacatccttcttcgaaatggaattgaacgttttcggggaacatcactgagcttctctgataaagagggaattcaaccatttcaggaatattcatatcagctgaaagcttgcacggttgctggctgtgccaccagtagcaaggtagttgcagctactacccaaggagttccggagagcatcctgccaccaagcatcacagccctaagtgcagtggctctgcatctgagctggagtgtccctgagaaatcaaacggcgtcattaaagagtaccagatcaggcaggttgggaaaggtctcatccacactgacaccactgacaggagacagcatacggtcacaggtctccagccatacaccaactacagcttcactcttacagcttgtacatctgctgggtgcacttcaagcgagccttttctaggtcagacactgcaggcagctcctgaaggagtttgggtgacacctcgacacattatcatcaattctacaacagtggaattatattggagtctgccagaaaagcccaatggcctcgtttctcaatatcaattgagtcgtaatggaaacttgcttttcctgggtggcagtgaggagcagaatttcactgataaaaacctggagcccaatagcagatacacttacaagttagaagtcaaaactggaggtggcagcagtgctagtgatgattacattgttcaaacacctatgtcaacaccagaagaaatctatcctccatataatatcacagtaattgggccttattctatatttgtagcttggataccaccagggatcctcatccccgaaattcctgtggagtacaatgtcttactcaatgatggaagtgtaacacctctggccttctccgttggtcatcatcaatccacccttctggaaaatttgactccattcacacagtatgagataaggatacaagcatgtcaaaatggaagttgtggagttagcagtaggatgtttgtcaaaacacctgaagcagccccaatggatcttaattctcctgttcttaaggcactggggtcagcttgcatagagattaagtggatgccacctgaaaaaccaaatggaatcatcatcaactactttatttacagacgccctgctggcattgaagaggagtctgttttatttgtctggtcagaaggagcccttgaatttatggatgaaggagacaccctgaggcctttcacactctacgaatatcgggtcagagcctgtaactccaagggttcagtggagagtctgtgggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctg

SEQ ID NO: 43 - protein, 913 aa, Artificial Sequence
  MiniUSH2A-3
MNCPVLSLGSGFLFQVIEMLIFAYFASISLTESRGLFPRLENVGAFKSAGLHATTCDGEPQGMLPPEVVIINSTAVRVIWTSPSNPNGVVTEYSIYVNNKLYKTGMNVPGSFILRDLSPFTIYDIQVEVCTIYACVKSNGTQITTVEDTPSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRGLSKAVRARTKEDVPQGVSPPTWTKIDNLEDTIVLNWRKPIQSNGPIIYYILLRNGIERFRGTSLSFSDKEGIQPFQEYSYQLKACTVAGCATSSKVVAATTQGVASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 44 - DNA, 2739 bp, Artificial Sequence
  MiniUSH2A-3
atgaattgcccagttctttcattgggctctggcttcttgtttcaggtcattgaaatgttgatctttgcctattttgcttcaatatccttgactgagtcacgaggtcttttcccaaggctggagaacgtgggagctttcaagagtgcaggacttcatgcaaccacttgcgatggggagcctcagggcatgcttcctccagaggttgtcatcatcaacagtacagctgtacgtgtcatctggacatctccttcaaacccaaatggtgttgtcactgagtattctatctatgtaaataataagctctacaagactggaatgaatgtgcctgggtcgtttattctgagagacctgtctcccttcactatctatgacattcaggttgaagtctgcacaatatatgcctgcgtgaaaagcaatggaacccaaattaccactgtggaagacactccaagtgatataccaacacccacaattcgtggcatcacttcaagatctcttcaaattgattgggtgtctccacggaagccaaatggcatcattcttggatatgatctcctatggaaaacatggtatccatgcgctaaaactcaaaagttagtgcaggatcagagtgatgagctctgcaaggcagtgaggtgtcaaaaacctgaatctatctgtggacacatttgctattcttctgaagctaaggtttgttgtaacggagtgctctataaccccaagcctggacatcgctgttgtgaagaaaagtatatcccgtttgttctgaattctactggagtttgttgtggtggccgaatacaggaggcacaaccaaatcatcagtgctgctctgggtattacgctagaattctaccaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtatgttctgttgtgggcaggattatgtgaatatgtcagataccatatgctgctcagcttccagtggagagtctaaagcacatattaaaaagaatgacccggtgccagtaaaatgctgtgagactgaacttattccaaagagccagaaatgctgtaatggagttggatataatcctttgaaatatgtttgctctgacaagatttcaactggaatgatgatgaaggaaaccaaagagtgcaggatcctctgcccagcatctatggaagccacagaacattgtggcaggtgtgacttcaactttaccagccacatttgcactgtgataagagggtctcacaattccacagggaaggcatcaattgaagaaatgtgttcatctgccgaagaaaccattcatacagggagtgtaaacacgtactcttacacagatgtgaacctcaagccctacatgacatatgagtacaggatttctgcctggaacagctatgggcgaggactcagcaaagctgtgagagccagaacaaaagaagatgtgcctcaaggagtgagtccccctacgtggaccaaaatagacaatcttgaagatacaattgtcttaaactggagaaaacctatacaatcaaatggtcctattatttactacatccttcttcgaaatggaattgaacgttttcggggaacatcactgagcttctctgataaagagggaattcaaccatttcaggaatattcatatcagctgaaagcttgcacggttgctggctgtgccaccagtagcaaggtagttgcagctactacccaaggagttgcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctg

SEQ ID NO: 45 - protein, 435 aa, Artificial Sequence
  MiniUSH2A-4
MNCPVLSLGSGFLFQVIEMLIFAYFASISLTESRGLFPRLENVGAFKSKGPTAELRTHPAPPSGLSSPQIGTLASRTASFRWSPPMFPNGVIHSYELQFHVACPPDSALPCTPSQIETKYTGLGQKASLGGLQPYTTYKLRVVAHNEVGSTASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 46 - DNA, 1305 bp, Artificial Sequence
  MiniUSH2A-4
atgaattgcccagttctttcattgggctctggcttcttgtttcaggtcattgaaatgttgatctttgcctattttgcttcaatatccttgactgagtcacgaggtcttttcccaaggctggagaacgtgggagctttcaagagcaaaggaccgacagctgaactgagaacccatcctgccccaccctcaggactgtcctctccacaaatcgggacgctggcctcaaggacggcctccttccggtggagtccccccatgttccccaatggtgtcattcacagctatgaactccaattccacgtggcttgccctcctgactcagccctcccctgtactcccagccaaatagaaacaaagtacacggggctggggcagaaagccagccttgggggtctccagccctacaccacatacaagctgagagtggtggcacacaacgaggtgggcagtacggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctg

SEQ ID NO: 47 - protein, 331 aa, Artificial Sequence
  MiniUSH2A-5
MNCPVLSLGSGFLFQVIEMLIFAYFASISLTESRGLFPRLENVGAFKASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 48 - DNA, 993 bp, Artificial Sequence
  MiniUSH2A-5
atgaattgcccagttctttcattgggctctggcttcttgtttcaggtcattgaaatgttgatctttgcctattttgcttcaatatccttgactgagtcacgaggtcttttcccaaggctggagaacgtgggagctttcaaggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctg

SEQ ID NO: 49 - DNA, 33 bp, Artificial Sequence
  primer
aattcgagctcggtacatgaattgcccagttct

SEQ ID NO: 50 - DNA, 24 bp, Artificial Sequence
  primer
cggctcggcttgaaagctcccacg

SEQ ID NO: 51 - DNA, 23 bp, Artificial Sequence
  primer
ctttcaagccgagccgagaagtg

SEQ ID NO: 52 - DNA, 23 bp, Artificial Sequence
  primer
tcggaagcccacagactctccac

SEQ ID NO: 53 - DNA, 23 bp, Artificial Sequence
  primer
gtctgtgggcttccgagtggatc

SEQ ID NO: 54 - DNA, 32 bp, Artificial Sequence
  primer
gccaagcttgcatgccttacaggtgggtgtct

SEQ ID NO: 55 - DNA, 60 bp, Artificial Sequence
  primer
ggggacaagtttgtacaaaaaagcaggcttcgccgccgccatgaattgcccagttctttc

SEQ ID NO: 56 - DNA, 48 bp, Artificial Sequence
  primer
ggggaccactttgtacaagaaagctgggtcttacaggtgggtgtctgt

SEQ ID NO: 57 - DNA, 33 bp, Artificial Sequence; identical to SEQ ID NO: 49
  primer

SEQ ID NO: 58 - DNA, 28 bp, Artificial Sequence
  primer
ggattgtaacatccaacatcattaaagc

SEQ ID NO: 59 - DNA, 27 bp, Artificial Sequence
  primer
ttggatgttacaatccgtcagctattt

SEQ ID NO: 60 - DNA, 26 bp, Artificial Sequence
  primer
cggctcggaccccgtgtaaatttaac

SEQ ID NO: 61 - DNA, 23 bp, Artificial Sequence
  primer
cacggggtccgagccgagaagtg

SEQ ID NO: 62 - DNA, 32 bp, Artificial Sequence; identical to SEQ ID NO: 54
  primer

SEQ ID NO: 63 - DNA, 60 bp, Artificial Sequence; identical to SEQ ID NO: 55
  primer

SEQ ID NO: 64 - DNA, 48 bp, Artificial Sequence; identical to SEQ ID NO: 56
  primer

SEQ ID NO: 65 - DNA, 20 bp, Artificial Sequence
  primer
agacactctgcagtattcac

SEQ ID NO: 66 - DNA, 20 bp, Artificial Sequence
  primer
cagaactgaatactttcagc

SEQ ID NO: 67 - DNA, 20 bp, Artificial Sequence
  primer
gagtcgtttgaggtagcaga

SEQ ID NO: 68 - DNA, 20 bp, Artificial Sequence
  primer
tgcctcgtttcttcacagtc

SEQ ID NO: 69 - DNA, 20 bp, Artificial Sequence
  primer
gagcccaatgaaagaactgg

SEQ ID NO: 70 - DNA, 22 bp, Artificial Sequence
  primer
gtcgtcccgtcacatttattac

SEQ ID NO: 71 - DNA, 23 bp, Artificial Sequence
  primer
atcatgcagtcctactctgacac

SEQ ID NO: 72 - protein, 93 aa, Artificial Sequence
  polypeptide fragment
PAPPSGLSSPQIGTLASRTASFRWSPPMFPNGVIHSYELQFHVACPPDSALPCTPSQIETKYTGLGQKASLGGLQPYTTYKLRVVAHNEVGST

SEQ ID NO: 73 - DNA, 279 bp, Artificial Sequence
  polynucleotide fragment
cctgccccaccctcaggactgtcctctccacaaatcgggacgctggcctcaaggacggcctccttccggtggagtccccccatgttccccaatggtgtcattcacagctatgaactccaattccacgtggcttgccctcctgactcagccctcccctgtactcccagccaaatagaaacaaagtacacggggctggggcagaaagccagccttgggggtctccagccctacaccacatacaagctgagagtggtggcacacaacgaggtgggcagtacg

SEQ ID NO: 74 - protein, 435 aa, Artificial Sequence; identical to SEQ ID NO: 45
  MiniUSH2A-6

SEQ ID NO: 75 - DNA, 1308 bp, Artificial Sequence
  MiniUSH2A-6
atgaattgcccagttctttcattgggctctggcttcttgtttcaggtcattgaaatgttgatctttgcctattttgcttcaatatccttgactgagtcacgaggtcttttcccaaggctggagaacgtgggagctttcaagagcaaaggaccgacagctgaactgagaacccatcctgccccaccctcaggactgtcctctccacaaatcgggacgctggcctcaaggacggcctccttccggtggagtccccccatgttccccaatggtgtcattcacagctatgaactccaattccacgtggcttgccctcctgactcagccctcccctgtactcccagccaaatagaaacaaagtacacggggctggggcagaaagccagccttgggggtctccagccctacaccacatacaagctgagagtggtggcacacaacgaggtgggcagtacggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctgtaa

SEQ ID NO: 76 - DNA, 33 bp, Artificial Sequence; identical to SEQ ID NO: 49
  primer

SEQ ID NO: 77 - DNA, 24 bp, Artificial Sequence
  primer
cctttgctcttgaaagctcccacg

SEQ ID NO: 78 - DNA, 23 bp, Artificial Sequence
  primer
ctttcaagagcaaaggaccgaca

SEQ ID NO: 79 - DNA, 32 bp, Artificial Sequence; identical to SEQ ID NO: 54
  primer

SEQ ID NO: 80 - DNA, 60 bp, Artificial Sequence; identical to SEQ ID NO: 55
  primer

SEQ ID NO: 81 - DNA, 48 bp, Artificial Sequence; identical to SEQ ID NO: 56
  primer

SEQ ID NO: 82 - DNA, 33 bp, Artificial Sequence; identical to SEQ ID NO: 49
  primer

SEQ ID NO: 83 - DNA, 24 bp, Artificial Sequence
  primer
tcggaagccttgaaagctcccacg

SEQ ID NO: 84 - DNA, 23 bp, Artificial Sequence
  primer
ctttcaaggcttccgagtggatc

SEQ ID NO: 85 - DNA, 32 bp, Artificial Sequence; identical to SEQ ID NO: 54
  primer

SEQ ID NO: 86 - DNA, 60 bp, Artificial Sequence; identical to SEQ ID NO: 55
  primer

SEQ ID NO: 87 - DNA, 48 bp, Artificial Sequence; identical to SEQ ID NO: 56
  primer

(19) United States
(12) Patent Application Publication
(10) Pub. No.: US 2021/0087583 A1
(43) Pub. Date: Mar. 25, 2021

(54) Minigene for the treatment of Usher syndrome type 2a and USH2A-associated retinitis pigmentosa.

(71) Applicant: Stichting Katholieke Universiteit, Nijmege (NL)

(72) Inventors: Hendrikus Antonius Rudolfus van Wyk, Nijmegen (NL); Johanna Maria Josephina Kremer, Oostrum (NP)

(73) Assignee: Stichting Katholieke Universiteit, Nijmegen (NL)

(21) Appl. No.: 16/970,635

(22) PCT Filed: Feb. 28, 2019
(86) PCT No.: PCT/EP2019/054984

§ 371 Date: Aug. 18, 2020

(30) Foreign Application Priority Data
Feb. 28, 2018 (EP) 18159185.0

(51) Int. Cl. C12N 15/86; C07K 14/78; A61K 31/7088

(52) U.S. Cl. CPC C12N 15/86; C07K 14/78; C12N 2740/13043; C12N 2710/10011; A61K 31/7088

(57) ABSTRACT

The present invention relates to the field of medicine. In particular, it relates to therapy for the treatment of Usher syndrome type 2a and USH2A-associated retinitis pigmentosa.

### FIELD OF THE INVENTION

The present invention relates to the field of medicine. In particular, it relates to therapy for the treatment of Usher syndrome type 2a and USH2A-associated retinitis pigmentosa.

### BACKGROUND OF THE INVENTION

Usher syndrome (USH) and non-syndromic retinitis pigmentosa (NSRP) are degenerative diseases of the retina. USH is clinically and genetically heterogeneous and by far the most common type of inherited deaf-blindness in man (1 in 20,000 individuals)(Kimberling et al, 2010). The hearing impairment in USH patients is mostly stable and congenital and can be partially compensated by providing patients with hearing aids or cochlear implants. NSRP is more prevalent than USH, occurring in 1 per 4,000 individuals (Hartong et al, 2006). The degeneration of photoreceptor cells in USH and NSRP is progressive and often leads to complete blindness between the fifth and seventh decade of life, thereby leaving time for therapeutic intervention. Mutations in the USH2A gene are the most frequent cause of USH explaining up to 50% of all USH patients worldwide (±500 patients in The Netherlands) and, as indicated by McGee et al (2010), also the most prevalent cause of NSRP in the USA (likely accounting for 12-25% of all cases of retinitis pigmentosa (RP); ±600 patients in The Netherlands). The mutations are spread throughout the 72 USH2A exons and their flanking intronic sequences, and consist of nonsense and missense mutations, deletions, duplications, large rearrangements, and variants affecting splicing (USHbases and unpublished results). USH and other retinal dystrophies, for long have been considered as incurable disorders. Despite the broad clinical potential of antisense oligonucleotide (AON)-based therapy, it is not frequently used in the vertebrate eye. In addition, antisense therapy for exon skipping, when effective, only addresses mutations in specific exons. In that respect gene augmentation therapy would be a way to address more or even all mutations. Recent and ongoing phase I/II clinical trials using gene augmentation therapy have led to promising results in selected groups of patients with Leber Congenital Amaurosis and Usher syndrome due to mutations in the RPE65 (Bainbridge et al, 2008; Cideciyan et al, 2008; Hauswirth et al, 2008; Maguire et al, 2008) and MYO7A (Hashimoto et al, 2007; Lopes et al, 2013; Colella et al, 2014; Zallocchi et al, 2014) genes, respectively. The size of the coding sequence (15,606 bp) and the presence of multiple alternatively spliced transcripts with unknown significance, hamper gene augmentation therapy, due to the currently limiting cargo size of many available vectors (e.g. adeno-associated (AAV) and lentiviral vectors). There is thus a need for a condensed USH2A gene that can be fitted into a proper vector and can be used for gene augmentation therapy.

### SUMMARY OF THE INVENTION

The invention provides for a polynucleotide construct comprising:
a signal sequence, preferably an USH2A signal sequence,
a polynucleotide encoding an USH2A transmembrane domain (TM), and
a polynucleotide encoding an USH2A intracellular region including the PDZ binding motif (PBM).

The invention further provides for a viral vector expressing a polynucleotide construct according to the invention.

The invention further provides for a pharmaceutical composition comprising the polynucleotide construct according to the invention or the viral vector according to the invention and a pharmaceutically acceptable excipient.

The invention further provides for the polynucleotide construct according to the invention, the vector according to the invention and the composition according to the invention for use as a medicament.

The invention further provides for the polynucleotide construct according to the invention, the vector according to the invention and the composition according to the invention for use in the treatment or prevention of USH2A-associated retinitis pigmentosa.

### BRIEF DESCRIPTION OF THE FIGURES

FIG. 1. Construction of miniUSH2A fragments and generation of Tg(3xPRE-1_-1.2ZOP:Hsa.miniUSH2A-1, -2, -5 and -6, EGFP, cmc12:EGFP); ush2a^{rmc1}.

(A) Schematic presentation of the domain architecture of human usherin's° B, miniUSH2A-1, miniUSH2A-2, miniUSH2A-5 and miniUSH2A-6. The fragments of usherin's° B that are encoded in the miniUSH2A genes are boxed. Tol2-based vectors containing an enhanced zebrafish opsin promoter (3xPRE-1_-1.2ZOP) driving the expression of miniUSH2A-1 (6786 bp) (B), miniUSH2A-2 (4125 bp) (C), miniUSH2A-5 (993 bp), miniUSH2A-6 (1305 bp) and IRES-EGFP in zebrafish photoreceptors, were generated. The vector further contains the heart-specific cmcl2 promoter driving the expression of EGFP.

(D-E) The miniUSH2A-containing plasmids were co-injected with Tol2 transposase mRNA into one-cell stage ush2a^{rmc1} embryos. At 4 dpf, heart-specific EGFP expression could be observed for which the larvae were selected.

FIG. 2. Analysis of Tol2-based miniUSH2A-1 and Tol2-based miniUSH2A-2 genomic insertions in transgenic F2 larvae.

(A) Genomic DNA of transgenic F2 larvae was fragmented and adaptor-ligated. Nested PCR and Sanger sequencing revealed that miniUSH2A-1 is incorporated in chromosome 15 (larvae 2, 3, 6 and 7, ˜250 bp fragment), in chromosome 18 (larvae 4, and 8, ˜1.1 kb fragment), or at both genomic loci (larvae 1 and 5).

(B) A single copy of miniUSH2A-2 was incorporated in chromosome 17 (˜300 bp fragment).

FIG. 3A. Localization of miniUSH2A-1 and -2 in the retina of transgenic zebrafish (5 dpf).

(A) Schematic presentation of a cone photoreceptor cell with the expected localization of centrin and miniUSH2A.

(B-C) In the transgenic zebrafish larvae, miniUSH2A-1 or -2 is detected using an anti-human usherin antibody (originally a red signal; spots in left column: B, and C), while in wild-type larvae

(D) and ush2a^{rmc1} mutants (E) no signal is observed. (n=14 for all groups, from 2 biological replicates). In all images the nuclei are stained with DAPI (originally a blue signal; grey shadows) and anti-centrin is used as a marker for the connecting cilium and basal body (originally a green signal; spots in middle column B′, C′, D′ and E′). In the right column (B″, C″, D″ and E″), the signals of usherin and centrin are merged) Scale bars: 5 μm.

FIG. 3B. Localization of miniUSH2A-5 and -6 in the retina of transgenic zebrafish (5 dpf).

(A) Schematic presentation of a photoreceptor cell with the expected localization of poc5 and miniUSH2A.

(B-C) In the transgenic zebrafish larvae, miniUSH2A-5 or -6 is detected using an anti-human usherin antibody (originally a red signal; spots in left column: B, and C and in the right column: B′″ and C″), (n=11 for miniUSH2A-5; n=20 for miniUSH2A-6, from 2 biological replicates). In all images the nuclei are stained with DAPI (originally a blue signal; grey shadows) and anti-poc5 is used as a marker for the connecting cilium and basal body (originally a green signal; spots in the second column B′ and C′, and right column B′″ and C′″). In the third column (B″ and C″, and enlarged in the right column B′″ and C′″), the signals of usherin and poc5 are merged.

FIG. 4. Association of miniUSH2A-1 and -2 with Whrna.

(A) Whrna labeling (originally a red signal; spots in left column) at the photoreceptor periciliary region was significantly decreased in ush2a^{rmc1} larvae as compared to wild-type larvae (5 dpf). In transgenic larvae expressing miniUSH2A-1 and miniUSH2A-2, Whrna labeling at the periciliary region was restored (5 dpf). (n=14 larvae for each group from 2 biological replicates). Nuclei are counterstained with DAPI (originally a blue signal; grey shadows), and anti-centrin (originally a green signal: spots in middle column) was used as a basal body and connecting cilium marker. Scale bars: 10 μm.

(B) Quantification of Whrna localization (originally a red signal; spots in left column) at the photoreceptor periciliary region in both transgenic zebrafish lines as compared to wild-type and ush2a^{rmc1} larvae. Each single data point in the scatter graph displays the averaged mean grey value from the eye of one larva. (* indicates P<0.05, two-tailed unpaired Student's t-test). (C) GST pull down assay, showing that HA-tagged zebrafish Whrna was efficiently pulled down by GST-fused usherin (aa5064-aa5202), but not by GST alone. The third line shows 5% input of the protein extract.

FIG. 5. Visual Motor Responses in transgenic zebrafish expressing miniUSH2A-1 or -2 (5 dpf).

The eye-specific Light-ON Visual Motor Response (VMR) presented as the maximum velocity (mm/s) is shown for the time frame of 30 seconds prior and after light alternation. The average Vmax of wild-type larvae (originally a red line; TLF), ush2a^{rmc1} larvae (originally a blue line; ush2a^{rmc1}), miniUSH2A-1-expressing ush2a^{rmc1} larvae (black line; miniUSH2A 1), miniUSH2A-2-expressing ush2a^{rmc1} larvae (originally a green line; miniUSH2A 2) is shown. A clear increase in VMR is observed in both miniUSH2A-1 and miniUSH2A-2-expressing ush2a^{rmc1} larvae as compared to ush2a^{rmc1} mutants (5 dpf; n=56 minimum per group; minimum of 2 biological replicates).

FIG. 6A. Physiological rescue potential of miniUSH2A-1 and miniUSH2A-2.

(A) The average normalized b-wave amplitude (μV) was significantly reduced in ush2a^{rmc1} mutants as compared to strain-matched wild-type larvae (TLF, 5dpf). B-wave amplitudes recorded in ush2a^{rmc1} larvae expressing miniUSH2A-1 or miniUSH2A-2 were significantly improved as compared to ush2a^{rmc1} larvae.

(B) Statistical analysis of the maximum b wave amplitudes was performed using at least 13 larvae per experiment. * p<0.05; two-tailed unpaired Student's t-test.) (p<0.05; two-tailed unpaired Student's t-test; n=13 wild-type, n=21 ush2a^{rmc1}, n=27 miniUSH2A-1 and n=13 miniUSH2A 2 larvae, from minimal 2 biological replicates). Left column: TLF; second column from left: ush2a^{rmc1}; third column from left: miniUSH2A-1; right column: miniUSH2A-2.

FIG. 6B. Physiological rescue potential of miniUSH2A-5 and miniUSH2A-6.

(A) The average normalized b-wave amplitude (μV) was reduced in GFP-negative ush2a^{rmc1} mutants as compared to strain-matched wild-type larvae (WT TLF, 6dpf). B-wave amplitudes recorded in ush2a^{rmc1} larvae expressing miniUSH2A-6 were improved as compared to clutch-matched GFP-negative ush2a^{rmc1} larvae.

(B) Dot plot of the maximum b wave amplitudes of individual larvae (n=10 WT TLF, n=9 ush2a^{rmc1}, n=9 miniUSH2A-6).

(C) The average normalized b-wave amplitude (μV) was significantly reduced in GFP-negative ush2a^{rmc1} mutants as compared to strain-matched wild-type larvae (WT TLF, 6 dpf) (one-way ANOVA Tukey's Multiple Comparison Test (* P<0.05)). B-wave amplitudes recorded in ush2a^{rmc1} larvae expressing miniUSH2A-5 were improved as compared to clutch-matched GFP-negative ush2a^{rmc1} larvae.

(D) Dot plot of the maximum b wave amplitudes of individual larvae (n=11 WT TLF, n=11 ush2a^{rmc1}, n=11 miniUSH2A-5).

### DESCRIPTION OF THE SEQUENCES

| SEQ ID NO: | Name: | Type: |
|---|---|---|
| 1 | USH2A wild-type | PRT |
| 2 | USH2A wild-type | CDS |
| 3 | USH2A signal sequence | PRT |
| 4 | USH2A signal sequence | CDS |
| 5 | USH2A transmembrane domain (TM) | PRT |
| 6 | USH2A transmembrane domain (TM) | CDS |
| 7 | USH2A intracellular region including the PDZ | PRT |
| | binding motif (PBM) | |
| 8 | USH2A intracellular region including the PDZ | CDS |
| | binding motif (PBM) | |
| 9 | USH2A fibronectin 3 domain (FN3)_1 (aa 2925- | PRT |
| | 3007 of wild-type) | |
| 10 | USH2A fibronectin 3 domain (FN3)_1 (aa 2925- | CDS |
| | 3007 of wild-type) | |
| 11 | USH2A fibronectin 3 domain (FN3)_2 (aa 3020- | PRT |
| | 3096 of wild-type) | |
| 12 | USH2A fibronectin 3 domain (FN3)_2 (aa 3020- | CDS |
| | 3096 of wild-type) | |
| 13 | USH2A fibronectin 3 domain (FN3)_3 (aa 3502- | PRT |
| | 3576 of wild-type) | |
| 14 | USH2A fibronectin 3 domain (FN3)_3 (aa 3502- | CDS |
| | 3576 of wild-type) | |
| 15 | USH2A fibronectin 3 domain (FN3)_4 (aa 3590- | PRT |
| | 3667 of wild-type) | |
| 16 | USH2A fibronectin 3 domain (FN3)_4 (aa 3590- | CDS |
| | 3667 of wild-type) | |
| 17 | USH2A fibronectin 3 domain (FN3)_5 (aa 3681- | PRT |
| | 3758 of wild-type) | |
| 18 | USH2A fibronectin 3 domain (FN3)_5 (aa 3681- | CDS |
| | 3758 of wild-type) | |
| 19 | USH2A fibronectin 3 domain (FN3)_6 (aa 3772- | PRT |
| | 3855 of wild-type) | |
| 20 | USH2A fibronectin 3 domain (FN3)_6 (aa 3772- | CDS |
| | 3855 of wild-type) | |
| 21 | USH2A fibronectin 3 domain (FN3)_7 (aa 3864- | PRT |
| | 3951 of wild-type) | |
| 22 | USH2A fibronectin 3 domain (FN3)_7 (aa 3864- | CDS |
| | 3951 of wild-type) | |
| 72 | USH2A fibronectin 3 domain (FN3)_32 (aa 4826- | PRT |
| | 4918 of wild-type) | |
| 73 | USH2A fibronectin 3 domain (FN3)_32 (aa 4826- | CDS |
| | 4918 of wild-type) | |
| 23 | USH2A cysteine-rich fibronectin 3 domain | PRT |
| 24 | USH2A cysteine-rich fibronectin 3 domain | CDS |
| 25 | USH2A laminin G-like domain (LamGL) | PRT |
| 26 | USH2A laminin G-like domain (LamGL) | CDS |
| 27 | USH2A laminin N-terminal domain (LamNT) | PRT |
| 28 | USH2A laminin N-terminal domain (LamNT) | CDS |
| 29 | USH2A laminin-type EGF-like domain (EGF | PRT |
| | Lam)_1 (aa 518-572 of wild-type) | |
| 30 | USH2A laminin-type EGF-like domain (EGF | CDS |
| | Lam) 1 (aa 518-572 of wild-type) | |
| 31 | USH2A laminin-type EGF-like domain (EGF | PRT |
| | Lam)_2 (aa 575-638 of wild-type) | |
| 32 | USH2A laminin-type EGF-like domain (EGF | CDS |
| | Lam)_2 (aa 575-638 of wild-type) | |
| 33 | USH2A laminin-type EGF-like domain (EGF | PRT |
| | Lam)_3 (aa 641-691 of wild-type) | |
| 34 | USH2A laminin-type EGF-like domain (EGF | CDS |
| | Lam)_3 (aa 641-691 of wild-type) | |
| 35 | USH2A laminin-type EGF-like domain (EGF | PRT |
| | Lam)_4 (aa 694-744 of wild-type) | |
| 36 | USH2A laminin-type EGF-like domain (EGF | CDS |
| | Lam)_4 (aa 694-744 of wild-type) | |
| 37 | USH2A laminin G domain (LamG) | PRT |
| 38 | USH2A laminin G domain (LamG) | CDS |
| 39 | MiniUSH2A-1 | PRT |
| 40 | MiniUSH2A-1 | CDS |
| 41 | MiniUSH2A-2 | PRT |
| 42 | MiniUSH2A-2 | CDS |
| 43 | MiniUSH2A-3 | PRT |
| 44 | MiniUSH2A-3 | CDS |
| 45 | MiniUSH2A-4 | PRT |
| 46 | MiniUSH2A-4 | CDS |
| 47 | MiniUSH2A-5 | PRT |
| 48 | MiniUSH2A-5 | CDS |
| 74 | MiniUSH2A-6 | PRT |
| 75 | MiniUSH2A-6 | CDS |
| 49 | PCR primer | DNA |
| 50 | PCR primer | DNA |
| 51 | PCR primer | DNA |
| 52 | PCR primer | DNA |
| 53 | PCR primer | DNA |
| 54 | PCR primer | DNA |
| 55 | PCR primer | DNA |
| 56 | PCR primer | DNA |
| 57 | PCR primer | DNA |
| 58 | PCR primer | DNA |
| 59 | PCR primer | DNA |
| 60 | PCR primer | DNA |
| 61 | PCR primer | DNA |
| 62 | PCR primer | DNA |
| 63 | PCR primer | DNA |
| 64 | PCR primer | DNA |
| 65 | PCR primer | DNA |
| 66 | PCR primer | DNA |
| 67 | PCR primer | DNA |
| 68 | PCR primer | DNA |
| 69 | PCR primer | DNA |
| 70 | PCR primer | DNA |
| 71 | PCR primer | DNA |
| 76 | PCR primer | DNA |
| 77 | PCR primer | DNA |
| 78 | PCR primer | DNA |
| 79 | PCR primer | DNA |
| 80 | PCR primer | DNA |
| 81 | PCR primer | DNA |
| 82 | PCR primer | DNA |
| 83 | PCR primer | DNA |
| 84 | PCR primer | DNA |
| 85 | PCR primer | DNA |
| 86 | PCR primer | DNA |
| 87 | PCR primer | DNA |

### DETAILED DESCRIPTION OF THE INVENTION

The inventors have arrived at the surprising finding that a minigene can be constructed for the treatment by gene augmentation of USH2A-associated retinitis pigmentosa and Usher syndrome. The minigene according to the invention encodes a sufficient part of the USH2A polypeptide in order to confer effective treatment.

Accordingly, in a first aspect the invention provides for a polynucleotide construct comprising:
a polynucleotide encoding a signal sequence, preferably an USH2A signal sequence,
a polynucleotide encoding an USH2A transmembrane domain (TM), and
a polynucleotide encoding the USH2A intracellular region including the PDZ binding motif (PBM). Preferably, the polynucleotide construct does not encode a wild-type USH2A polypeptide and/or is not the wild-type polynucleotide according to SEQ ID NO: 2. Preferably, the polynucleotide construct does not encode the wild-type polypeptide according to SEQ ID NO: 1. Preferably, the polynucleotide construct has a length of at most 10 kbp, more preferably at most 9 kbp, more preferably at most 8 kbp, more preferably at most 7 kbp, more preferably at most 6 kbp, more preferably at most 5 kbp, more preferably at most 4.9, 4.8, or 4.7 kbp. Preferably the polynucleotide construct can be expressed in a viral vector, preferably an adeno associated viral vector (AAV).

The polynucleotide construct is herein referred to as the polynucleotide construct according to the invention. The term polynucleotide construct according to the invention is herein interchangeably used with the term minigene according to the invention. In all embodiments of the invention, the gene augmentation is to be construed as that a sufficient amount of the gene product of the minigene according to the invention is produced to confer improved function of the photoreceptor cells that are affected by an aberrant USH2A.

The signal sequence is herein referred to as a signal sequence according to the invention and may be any signal sequence that establishes that the immature protein is transferred to the ER (endoplasmic reticulum). A preferred signal sequence is the USH2A signal sequence. A preferred USH2A signal sequence has at least 50% sequence identity with SEQ ID NO: 3. A preferred polynucleotide encoding an USH2A signal sequence has at least 50% sequence identity with SEQ ID NO: 4.

The USH2A transmembrane domain (TM) is herein referred to as an USH2A transmembrane domain (TM) according to the invention. A preferred USH2A transmembrane domain (TM) has at least 50% sequence identity with SEQ ID NO: 5. A preferred polynucleotide encoding an USH2A transmembrane domain (TM) has at least 50% sequence identity with SEQ ID NO: 6.

The USH2A intracellular region including the PDZ binding motif (PBM) is herein referred to as an USH2A intracellular region including the PDZ binding motif (PBM) according to the invention. A preferred USH2A intracellular region including the PDZ binding motif (PBM) has at least 50% sequence identity with SEQ ID NO: 7. A preferred polynucleotide encoding an USH2A intracellular region including the PDZ binding motif (PBM) has at least 50% sequence identity with SEQ ID NO: 8.

Preferably, the polynucleotide construct according to the invention further comprises a polynucleotide encoding an USH2A fibronectin 3 domain (FN3). The USH2A fibronectin 3 domain (FN3) is herein referred to as the USH2A fibronectin 3 domain (FN3) according to the invention. A preferred USH2A fibronectin 3 domain (FN3) has at least 50% sequence identity with SEQ ID NO: 9.

The wild-type USH2A protein comprises 32 FN3 domains. Either of the 32 can be used in the polynucleotide construct according to the invention with a preference for domains SEQ ID NO: 9, 11, 13, 15, 17, 19, 21, 72, encoded by SEQ ID NO: 10, 12, 14, 16, 18, 20, 22, 73, respectively. In the embodiments of the invention, when more than one USH2A fibronectin 3 domain (FN3) is present, the domains are preferably the ones corresponding to in the sequence of the wild-type USH2A protein, such as FN3_1 up to FN3_7 and FN3_32 (SEQ ID NO: 9, 11, 13, 15, 17, 19, 21, 72, respectively). Preferably, the linker sequences of the wild-type protein are present as well. A preferred polynucleotide encoding an USH2A fibronectin 3 domain (FN3) has at least 50% sequence identity with SEQ ID NO: 10. In the embodiments of the invention, when more than one USH2A fibronectin 3 domain (FN3) is present, the polynucleotides encoding the domains are preferably the ones corresponding to the sequence of the wild-type USH2A polynucleotide, such as FN3_1 up to FN3_7 and FN3_32 (SEQ ID NO: 10, 12, 14, 16, 18, 20, 22, 73, respectively).

Preferably, the linker sequences of the wild-type polynucleotide are present as well. The person skilled in the art knows how to identify the protein and polynucleotide domains and linkers in the wild-type sequences (SEQ ID NO: 1 and 2, respectively).

Preferably, the polynucleotide construct according to the invention further comprises a polynucleotide encoding an USH2A cysteine-rich fibronectin 3 domain. The USH2A cysteine-rich fibronectin 3 domain is herein referred to as an USH2A cysteine-rich fibronectin 3 domain according to the invention. A preferred USH2A cysteine-rich fibronectin 3 domain has at least 50% sequence identity with SEQ ID NO: 23. A preferred polynucleotide encoding an USH2A cysteine-rich fibronectin 3 domain has at least 50% sequence identity with SEQ ID NO: 24. Preferably, the polynucleotide construct according to the invention comprises at least two USH2A fibronectin 3 domains (FN3) according to the invention. In an embodiment, the polynucleotide construct according to the invention comprises two polynucleotides encoding an USH2A fibronectin 3 domain (FN3) according to the invention. More preferably, the polynucleotide construct according to the invention comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 polynucleotides encoding an USH2A fibronectin 3 domain (FN3) according to the invention. In an embodiment, the polynucleotide construct according to the invention comprises seven polynucleotides encoding an USH2A fibronectin 3 domain (FN3) according to the invention.

Preferably, the polynucleotide construct according to the invention further comprises a polynucleotide encoding a domain selected from the group consisting of:
a polynucleotide encoding an USH2A laminin G-like domain (LamGL), a polynucleotide encoding an USH2A laminin N-terminal domain (LamNT), a polynucleotide encoding an USH2A laminin-type EGF-like domain (EGF Lam) and a polynucleotide encoding an USH2A laminin G domain (LamG).

The USH2A laminin G-like domain (LamGL) is herein referred to as an USH2A laminin G-like domain (LamGL) according to the invention. A preferred USH2A laminin G-like domain (LamGL) has at least 50% sequence identity with SEQ ID NO: 25. A preferred polynucleotide encoding an USH2A laminin G-like domain (LamGL) has at least 50% sequence identity with SEQ ID NO: 26. The USH2A laminin N-terminal domain (LamNT) is herein referred to as an USH2A laminin N-terminal domain (LamNT) according to the invention. A preferred USH2A laminin N-terminal domain (LamNT) has at least 50% sequence identity with SEQ ID NO: 27. A preferred polynucleotide encoding an USH2A laminin N-terminal domain (LamNT) has at least 50% sequence identity with SEQ ID NO: 28.

The USH2A laminin-type EGF-like domain (EGF Lam) is herein referred to as an USH2A laminin-type EGF-like domain (EGF Lam) according to the invention. A preferred USH2A laminin-type EGF-like domain (EGF Lam) has at least 50% sequence identity with SEQ ID NO: 29. A preferred polynucleotide encoding an USH2A laminin-type EGF-like domain (EGF Lam) has at least 50% sequence identity with SEQ ID NO: 30. The wild-type USH2A protein comprises 10 EGF Lam domains. Either of the 10 can be used in the polynucleotide construct according to the invention with a preference for domains SEQ ID NO: 29, 31, 33, 35, encoded by SEQ ID NO: 30, 32, 34, 36, respectively.

In the embodiments of the invention, when more than one laminin-type EGF-like domain (EGF Lam) is present, the domains are preferably the ones corresponding to in the sequence of the wild-type USH2A protein, such as EGF Lam_1 up to EGF Lam_4 (SEQ ID NO: 29, 31, 33, 35, respectively). Preferably, the linker sequences of the wild-type protein are present as well.

In the embodiments of the invention, when more than one USH2A fibronectin 3 domain (FN3) is present, the polynucleotides encoding the domains are preferably the ones corresponding to the sequence of the wild-type USH2A polynucleotide, such as EGF Lam_1 up to EGF Lam_4 (SEQ ID NO: 30, 32, 34, 36, respectively). Preferably, the linker sequences of the wild-type polynucleotide are present as well. The person skilled in the art knows how to identify the protein and polynucleotide domains and linkers in the wild-type sequences (SEQ ID NO: 1 and 2, respectively).

Preferably, the polynucleotide construct according to the invention comprises two, three, four, five, six, seven, eight, nine or ten polynucleotides encoding an USH2A laminin-type EGF-like domain (EGF Lam) according to the invention. In an embodiment, the polynucleotide construct according to the invention comprises four polynucleotides encoding an USH2A laminin-type EGF-like domain (EGF Lam). In an embodiment, the polynucleotide construct according to the invention comprises ten polynucleotides encoding an USH2A laminin-type EGF-like domain (EGF Lam). The USH2A laminin G domain (LamG) is herein referred to as an USH2A laminin G domain (LamG) according to the invention. A preferred USH2A laminin G domain (LamG) has at least 50% sequence identity with SEQ ID NO: 37. A preferred polynucleotide encoding an USH2A laminin G domain (LamG) has at least 50% sequence identity with SEQ ID NO: 38. In an embodiment, the polynucleotide construct according to the invention comprises two polynucleotides encoding an USH2A laminin G domain (LamG). The wild-type USH2A protein comprises two LamG domains. Either of the two can be used in the polynucleotide construct according to the invention with a preference for domain SEQ ID NO: 37, encoded by SEQ ID NO: 38.

Preferably, the polynucleotide construct according to the invention further comprises a polynucleotide encoding an USH2A laminin G-like domain (LamGL), a polynucleotide encoding an USH2A laminin N-terminal domain (LamNT), at least four polynucleotides encoding an USH2A laminin-type EGF-like domain (EGF Lam), and a polynucleotide encoding an USH2A laminin G domain (LamG).

In an embodiment, the polynucleotide construct according to the invention comprises:
a polynucleotide encoding a signal sequence according to the invention,
a polynucleotide encoding an USH2A transmembrane domain (TM) according to the invention,
a polynucleotide encoding an USH2A intracellular region including the PDZ binding motif (PBM) according to the invention,
a polynucleotide, encoding an USH2A cysteine-rich fibronectin 3 domain, and
seven polynucleotides encoding an USH2A fibronectin 3 domain (FN3) according to the invention.

In an embodiment, the polynucleotide construct according to the invention comprises:
a polynucleotide encoding a signal sequence according to the invention,
a polynucleotide encoding an USH2A transmembrane domain (TM) according to the invention,
a polynucleotide encoding an USH2A intracellular region including the PDZ binding motif (PBM) according to the invention,
a polynucleotide, encoding an USH2A cysteine-rich fibronectin 3 domain, and
two polynucleotides encoding an USH2A fibronectin 3 domain (FN3) according to the invention.

In an embodiment, the polynucleotide construct according to the invention comprises:
a polynucleotide encoding a signal sequence according to the invention,
a polynucleotide encoding an USH2A transmembrane domain (TM) according to the invention,
a polynucleotide encoding an USH2A intracellular region including the PDZ binding motif (PBM) according to the invention, and
a polynucleotide encoding an USH2A fibronectin 3 domain (FN3) according to the invention.

In an embodiment, the polynucleotide construct according to the invention comprises:
a polynucleotide encoding a signal sequence according to the invention,
a polynucleotide encoding an USH2A transmembrane domain (TM) according to the invention, and
a polynucleotide encoding an USH2A intracellular region including the PDZ binding motif (PBM) according to the invention.

In an embodiment, the polynucleotide construct according to the invention comprises:
a polynucleotide encoding a signal sequence according to the invention,
a polynucleotide encoding an USH2A transmembrane domain (TM) according to the invention,
a polynucleotide encoding an USH2A intracellular region including the PDZ binding motif (PBM) according to the invention,
a polynucleotide, encoding an USH2A cysteine-rich fibronectin 3 domain according to the invention,
seven polynucleotides encoding an USH2A fibronectin 3 domain (FN3) according to the invention,
a polynucleotide encoding an USH2A laminin G-like domain (LamGL) according to the invention,
a polynucleotide encoding an USH2A laminin N-terminal domain (LamNT) according to the invention,
four polynucleotides encoding an USH2A laminin-type EGF-like domain (EGF Lam) according to the invention, and
a polynucleotide encoding an USH2A laminin G domain (LamG) according to the invention.

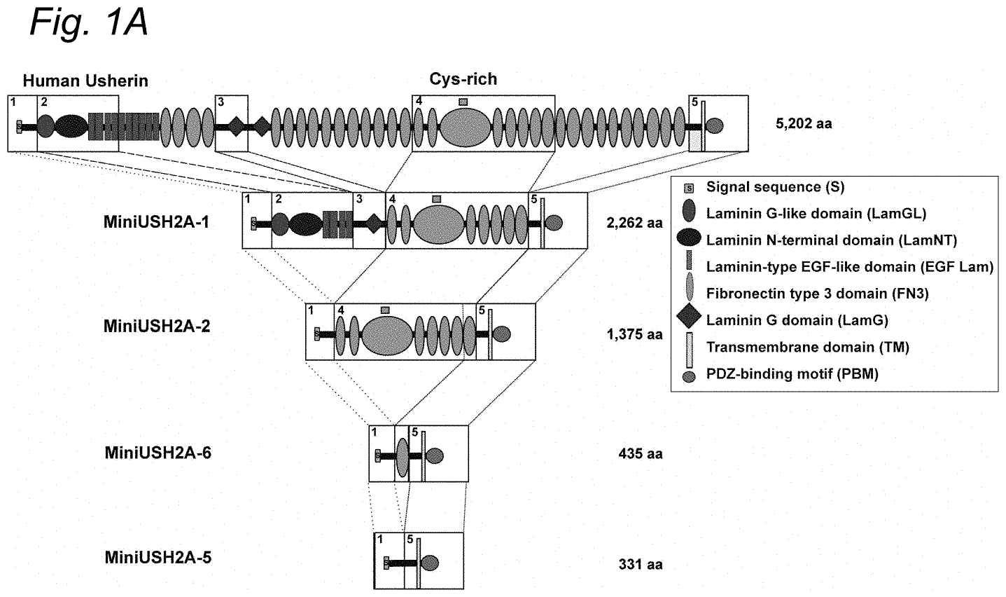

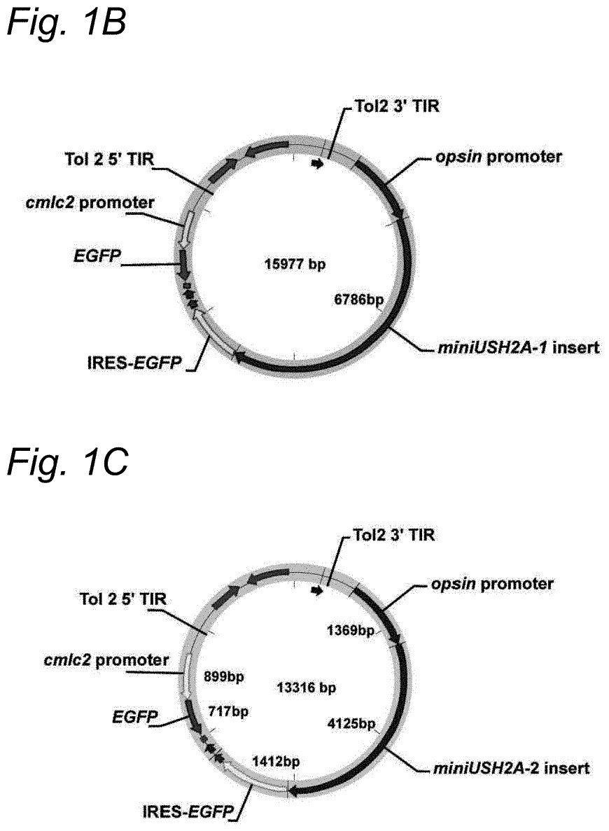

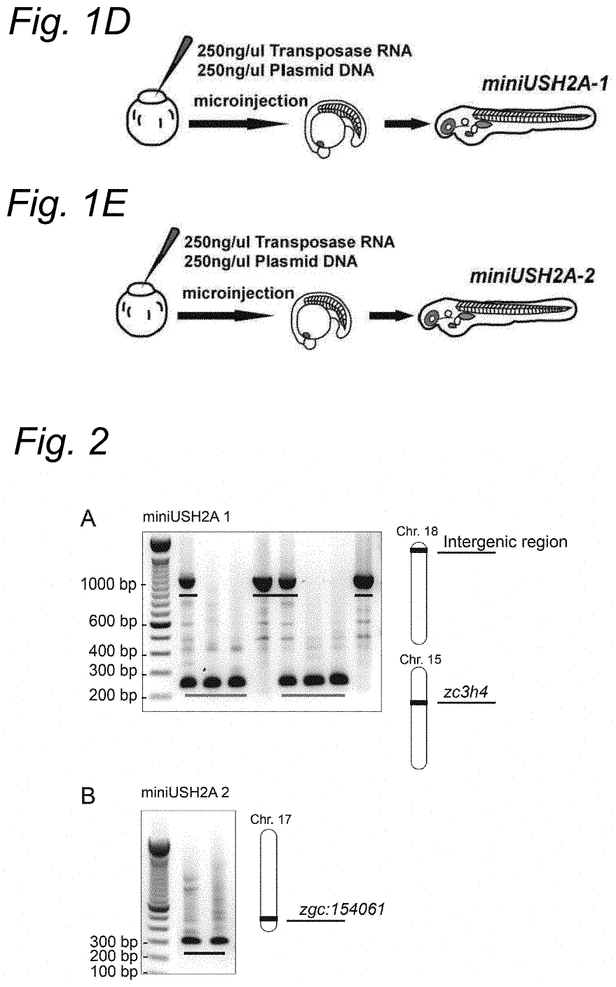

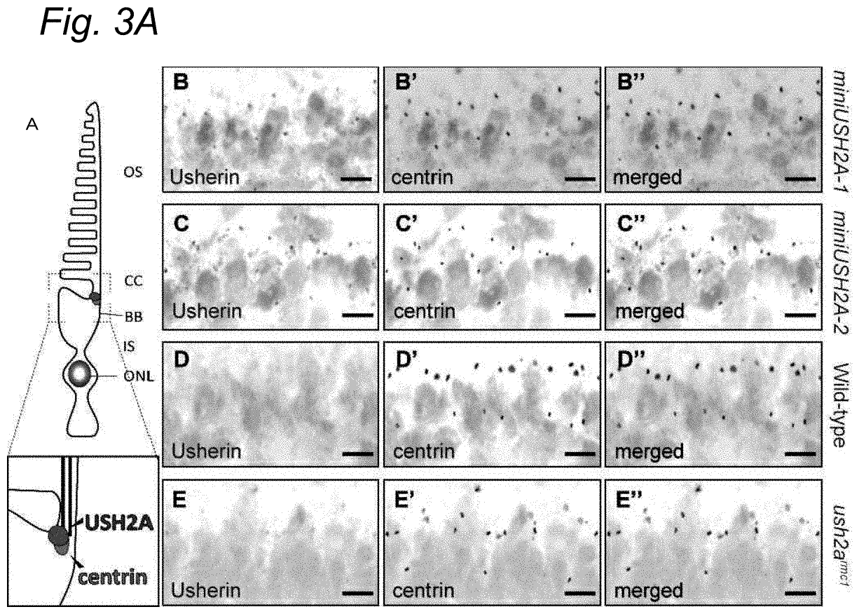

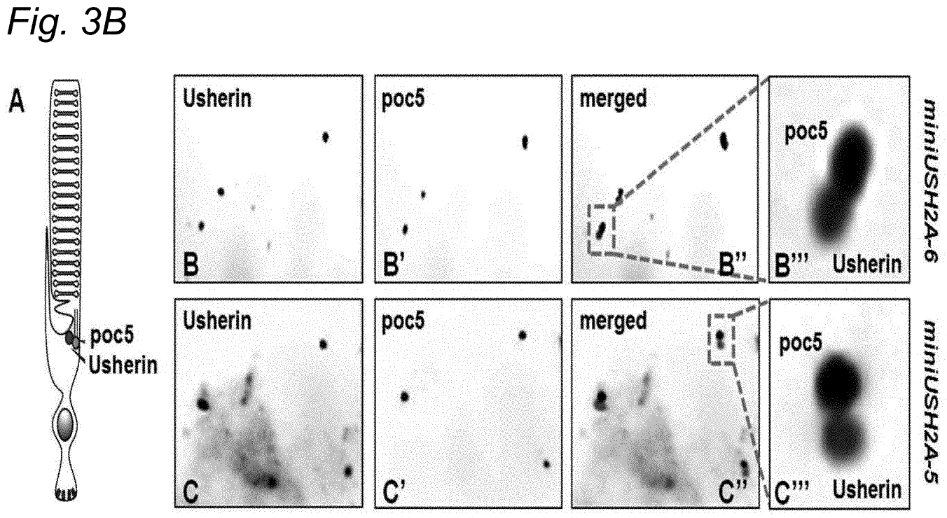

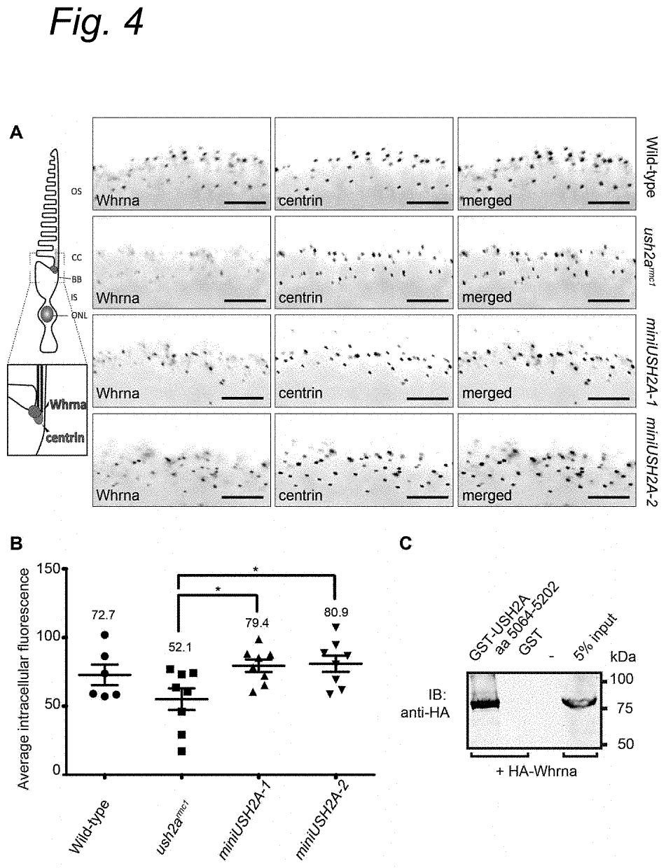

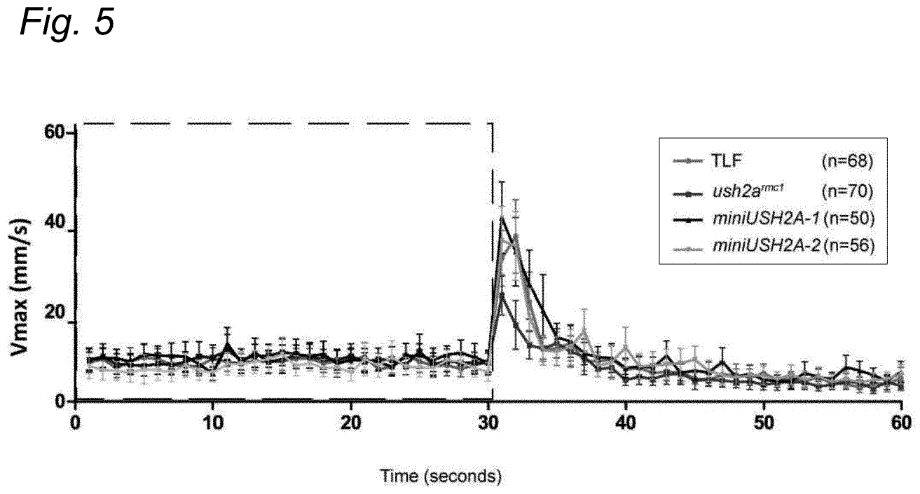

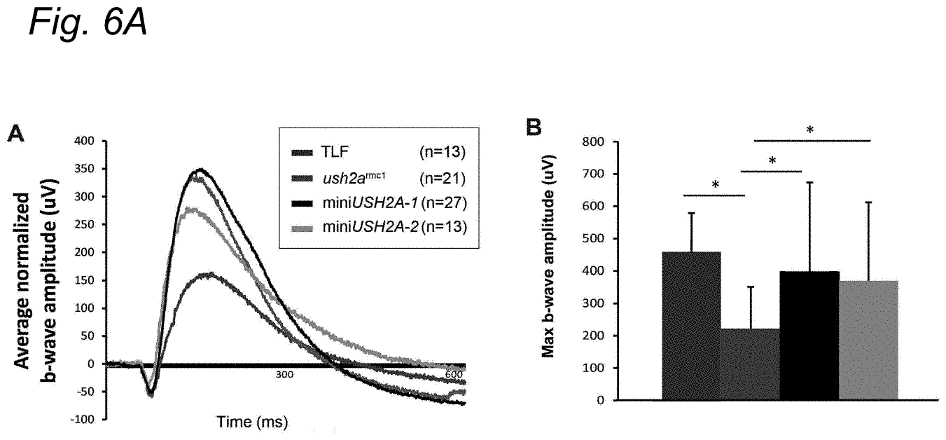

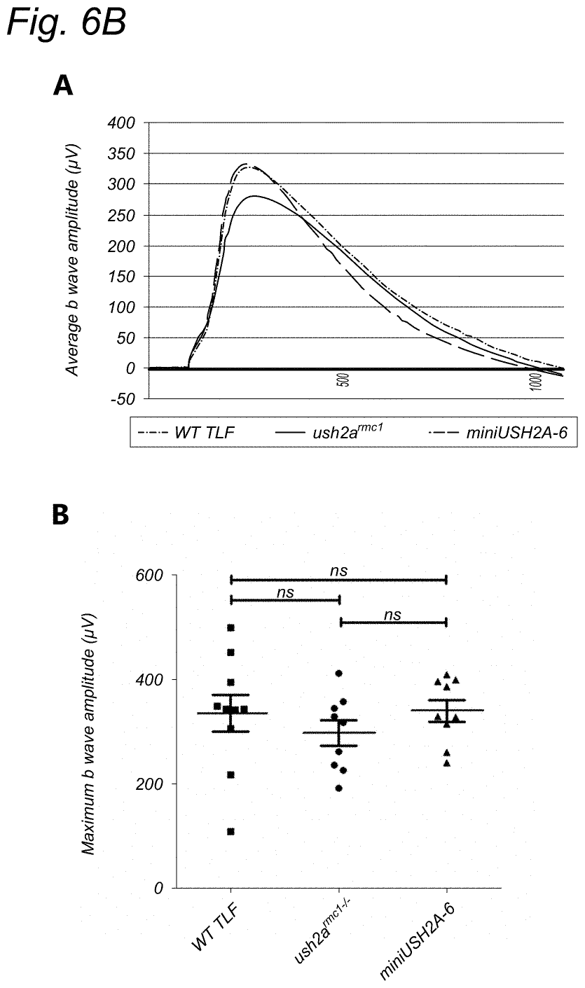

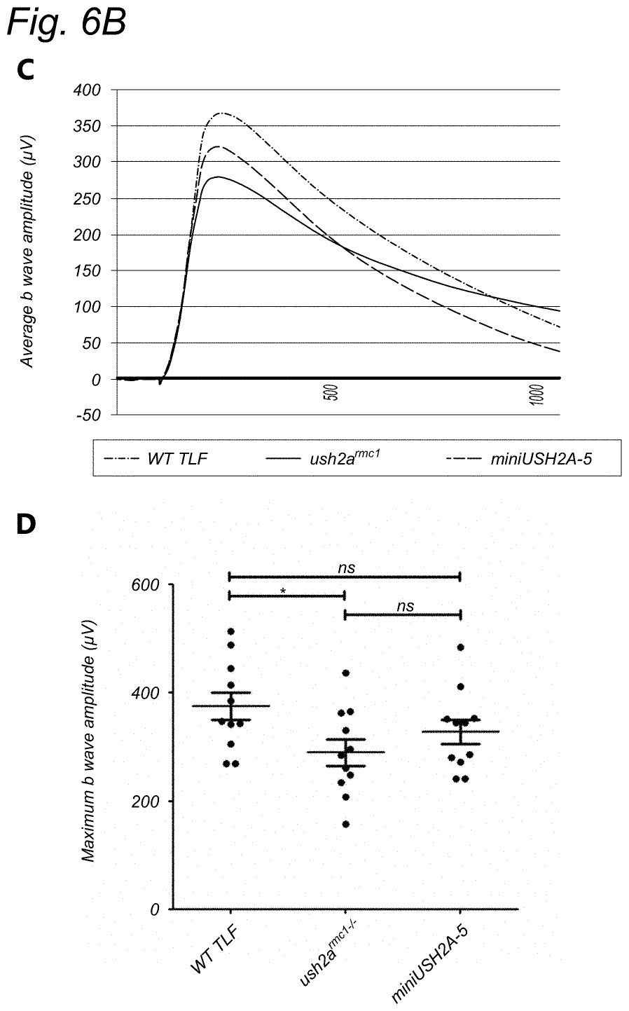

In an embodiment, the polynucleotide construct according to the invention encodes SEQ ID NO: 39 (MiniUSH2A-1). The encoded protein has preferably the genetic make-up as MiniUSH2A-1 in FIG. 1A.

In an embodiment, the polynucleotide construct according to the invention encodes SEQ ID NO:

41 (MiniUSH2A-2). The encoded protein has preferably the genetic make-up as MiniUSH2A-2 in FIG. 1A.

In an embodiment, the polynucleotide construct according to the invention encodes SEQ ID NO: 43 (MiniUSH2A-3).

In an embodiment, the polynucleotide construct according to the invention encodes SEQ ID NO: 45 (MiniUSH2A-4).

In an embodiment, the polynucleotide construct according to the invention encodes SEQ ID NO: 47 (MiniUSH2A-5).

In an embodiment, the polynucleotide construct according to the invention encodes SEQ ID NO:

74 (MiniUSH2A-6).

In an embodiment, the polynucleotide construct according to the invention has at least 50% sequence identity with SEQ ID NO: 40 (MiniUSH2A-1). The encoded protein has preferably the genetic make-up as MiniUSH2A-1 in FIG. 1A.

In an embodiment, the polynucleotide construct according to the invention has at least 50% sequence identity with SEQ ID NO: 42 (MiniUSH2A-2). The encoded protein has preferably the genetic make-up as MiniUSH2A-2 in FIG. 1A.

In an embodiment, the polynucleotide construct according to the invention has at least 50% sequence identity with SEQ ID NO: 44 (MiniUSH2A-3).

In an embodiment, the polynucleotide construct according to the invention has at least 50% sequence identity with SEQ ID NO: 46 (MiniUSH2A-4).

In an embodiment, the polynucleotide construct according to the invention has at least 50% sequence identity with SEQ ID NO: 48 (MiniUSH2A-5).

In an embodiment, the polynucleotide construct according to the invention has at least 50% sequence identity with SEQ ID NO: 75 (MiniUSH2A-6).

The polynucleotide construct according to the invention may comprise any further structural or non-structural and functional or non-functional polynucleotides or parts thereof that facilitate cloning or expression, such as linkers, restriction sites, cloning sites and the likes. Preferred further polynucleotides are those described elsewhere herein. In the embodiments of the invention, if linker sequences are used, these are preferably the linkers that are present in the wild-type USH2A protein and polynucleotide. The person skilled in the art will comprehend that some variation may be present in the linker(s) in view of the wild-type USH2A protein; it may be possible to shorten or lengthen linkers, insert heterologous and/or synthetic linkers, etcetera. In the embodiments of the invention, when multiple protein or polynucleotide domains are present, they are preferably present in the same order as in the wild-type protein and polynucleotide and may include the wild-type linker sequences.

Preferably, the polynucleotide construct according to the invention further comprises regulatory sequences that direct expression of the coding sequences in the polynucleotide construct. Such regulatory sequences are known to the person skilled in the art and include, but are not limited to, a promoter, a terminator and a Kozak sequence. Preferred regulatory sequences are those described in the examples herein.

In this aspect, there is also provided for a polypeptide encoded by any of the polynucleotides as defined here above, preferably a polypeptide with an amino acid sequence that has at least 50% sequence identity with SEQ ID NO: 3, 5, 7, 9, 11, 13, 15, 17, 19, 21, 23, 25, 27, 29, 30, 31, 33, 35, 37, 39, 41, 43, 45, 47, 72 or 74; more preferably a polypeptide with an amino acid sequence that has at least 50% sequence identity with SEQ ID NO: 39, 41, 43, 45, 47 or 74.

In a second aspect the invention provides for a vector comprising a polynucleotide construct according to the invention. Such vector may be any vector known to the person skilled in the art and include, but are not limited to, expression vectors, cloning vectors, subcloning vectors, nanoparticles, liposomes and viral vectors. All features of this aspect are preferably those of the first aspect.

A preferred viral vector is an adeno-associated viral vector (AAV) comprising the polynucleotide according to the invention, wherein the polynucleotide construct preferably further comprises an AAV inverted terminal repeat.

Another preferred viral vector is an lentiviral vector (LV) comprising the polynucleotide according to invention, wherein the polynucleotide construct preferably further comprises an LV long terminal repeat (LTR), preferably two LTRs.

A preferred AAV vector according to invention is a recombinant AAV vector and refers to an AAV vector comprising part of an AAV genome comprising an encoded exon skipping molecule according to the invention encapsulated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV5, AAV9 and others. Protein shell comprised of capsid protein may be derived from an AAV serotype such as AAV1, 2, 3, 4, 5, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention.

Preferably, a recombinant AAV vector according to the present invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the present invention comprises a capsid protein shell of AAV serotype 2 and the AAV genome or ITRs present in said vector are derived from AAV serotype 5; such vector is referred to as an AAV 2/5 vector.

More preferably, a recombinant AAV vector according to the present invention comprises a capsid protein shell of AAV serotype 2 and the AAV genome or ITRs present in said vector are derived from AAV serotype 8; such vector is referred to as an AAV 2/8 vector.

More preferably, a recombinant AAV vector according to the present invention comprises a capsid protein shell of AAV serotype 2 and the AAV genome or ITRs present in said vector are derived from AAV serotype 9; such vector is referred to as an AAV 2/9 vector.

More preferably, a recombinant AAV vector according to the present invention comprises a capsid protein shell of AAV serotype 2 and the AAV genome or ITRs present in said vector are derived from AAV serotype 2; such vector is referred to as an AAV 2/2 vector.

A preferred AAV-based vector comprises an expression cassette that is driven by a polymerase III-promoter (Pol III). A preferred Pol III promoter is, for example, a U1, a U6, or a U7 RNA promoter.

“AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73(2): 1309-1319) or U.S. Pat. No. 5,139,941, incorporated herein by reference. The AAV helper functions can be supplied on a AAV helper construct, which may be a plasmid. Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

Preferably, an AAV genome as present in a recombinant AAV vector according to the present invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

In a third aspect, the invention provides for a pharmaceutical composition comprising the polynucleotide construct according to the invention, the vector according to invention, the AAV according to the invention, or the LV according to the invention, further comprising a pharmaceutically acceptable excipient. The pharmaceutical composition is herein referred to as a pharmaceutical composition according to the invention. All features of this aspect are preferably those of the first and second aspect. Pharmaceutically acceptable excipients are known to the person skilled in the art. The person skilled in the art is able to select an appropriate pharmaceutically acceptable excipient.

In a fourth aspect, the invention provides for a method of treatment or prevention of USH2A-associated retinitis pigmentosa in a subject in need thereof, comprising administration of the polynucleotide construct according to the invention, the vector according to the invention, the AAV according to the invention, or the LV according to the invention to the subject.

In this aspect, the invention also provides for the polynucleotide construct according to the invention, the vector according to the invention, the AAV according to the invention, or the LV according to the invention for use as a medicament.

In this aspect, the invention also provides for the polynucleotide construct according to the invention, the vector according to the invention, the AAV according to the invention, or the LV according to the invention for use in the treatment or prevention of USH2A-associated retinitis pigmentosa in a subject in need thereof.

All features of this aspect are preferably those of the first, second and third aspect.

Unless otherwise indicated each embodiment as described herein may be combined with another embodiment as described herein.

### Definitions

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence. A “nucleic acid construct” is defined as a nucleic acid molecule which is isolated from a naturally occurring gene or which has been modified to contain segments of nucleic acids which are combined or juxtaposed in a manner which would not otherwise exist in nature. A nucleic acid molecule is represented by a nucleotide sequence. Optionally, a nucleotide sequence present in a nucleic acid construct is operably linked to one or more control sequences, which direct the production or expression of said peptide or polypeptide in a cell or in a subject.

“Operably linked” is defined herein as a configuration in which a control sequence is appropriately placed at a position relative to the nucleotide sequence coding for the polypeptide of the invention such that the control sequence directs the production/expression of the peptide or polypeptide of the invention in a cell and/or in a subject. “Operably linked” may also be used for defining a configuration in which a sequence is appropriately placed at a position relative to another sequence coding for a functional domain such that a chimeric polypeptide is encoded in a cell and/or in a subject.

“Expression” is construed as to include any step involved in the production of the peptide or polypeptide including, but not limited to, transcription, post-transcriptional modification, translation, post-translational modification and secretion.

A “control sequence” is defined herein to include all components which are necessary or advantageous for the expression of a polypeptide. At a minimum, the control sequences include a promoter and transcriptional and translational stop signals. Optionally, a promoter represented by a nucleotide sequence present in a nucleic acid construct is operably linked to another nucleotide sequence encoding a peptide or polypeptide as identified herein.

The term “transformation” refers to a permanent or transient genetic change induced in a cell following the incorporation of new DNA (i.e. DNA exogenous to the cell). When the cell is a bacterial cell, as is intended in the present invention, the term usually refers to an extrachromosomal, self-replicating vector which harbors a selectable antibiotic resistance.

An “expression vector” may be any vector which can be conveniently subjected to recombinant DNA procedures and can bring about the expression of a nucleotide sequence encoding a polypeptide of the invention in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context of the invention, a promoter preferably ends at nucleotide −1 of the transcription start site (TSS). A “polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The sequence information as provided herein should not be so narrowly construed as to require inclusion of erroneously identified bases. The skilled person is capable of identifying such erroneously identified bases and knows how to correct for such errors.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 5% of the value. Sequence identity herein of a polynucleotide, polynucleotide construct or of a polypeptide is preferably at least 50%. Preferably at least 50% is defined as preferably at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, at least 92%, at least 93%, at least 94%, at least 95%, at least 96%, at least 97%, more preferably at least 98%, more preferably at least 99%, or most preferably 100% sequence identity. In case of 100% sequence identity, the polynucleotide or polypeptide has exactly the sequence of the depicted SEQ ID NO:.

All patent and literature references cited in the present specification are hereby incorporated by reference in their entirety.

Unless otherwise indicated each embodiment as described herein may be combined with another embodiment as described herein.

The examples herein are offered for illustrative purposes only, and are not intended to limit the scope of the present invention in any way.

### Examples

Zebrafish maintenance and husbandry Experimental procedures were conducted in accordance with international and institutional guidelines (Dutch guidelines, protocol #RU-DEC 2012-301 and #RU-DEC 2016-0091). Wild type adult Tupfel Long-Fin (TLF) zebrafish and ush2a^{rmc1} mutants were used (c.2337_2342delinsAC; p.Cys780GInfsTer32). The zebrafish eggs were obtained from natural spawning of Tuebingen Long-Fin (TLF) breeding fish. Larvae were maintained and raised by standard methods (Kimmel et al, 1995).

Plasmid constructs Fragments encoding human usherin^{isoB} amino acid residues (aa) 1-744, aa 1682-1871, aa 2912-3955 and aa 4919-5202 (miniUSH2A-1) or, usherin^{isoB} aa 1-47, aa 2912-3955 and aa 4919-5202 (miniUSH2A-2), usherin^{isoB} aa 1-47 and aa 4815-5202 (miniUSH2A-6) or usherin^{isoB} aa 1-47 and aa 4919-5202 (miniUSH2A-5), were amplified from Human Retina Marathon®-Ready cDNA (Clontech, #639349) using Phusion® High-Fidelity DNA polymerase (New England Biolabs, #E0553), assembled and cloned in pUC19L using the GeneArt™ Seamless Cloning and Assembly Enzyme Mix (Thermo Fisher, #A14606) according to manufacturer's instructions (primers are listed in Table 1). Using Gateway® cloning technology the 3xPRE-ZOP promoter (kindly provided by Dr. Breandán Kennedy; Kennedy et al, 2001) was cloned in the pDONR™ P4-P1r vector in order to generate a p5'E vector. MiniUSH2A-1 and -2 were cloned in pDONR™221 in order to generate a pME vector. The p5'E-3xPRE-ZOP, pME-miniUSH2A-1 or -2 and p3'E-IRES-EGFPpA (Multisite Tol2kit clone 389; generously provided by Prof. Dr. Koichi Kawakami; Kwan et al, 2007) were cloned in the pDestTol2CG2 (Multisite Tol2kit clone 395) vector using the MultiSite Gateway® Three-Fragment Vector Construction Kit (Thermo Fisher, #12537-023), according to manufacturer's instruction.

TABLE 1
Primer sequences for the
construction of miniUSH2A-1 and -2

| | Subfragment | | SEQ |
|---|---|---|---|
| Minigene | primer name | Sequence | ID NO: |
| miniUSH2A- | pUC19L- | 5′-AATTCGAGCTCGGTACATGAATTGCCCAGTTCT-3′ | 49 |
| 2 | ss_fwd | | |
| miniUSH2A- | Ss- | 5′-CGGCTCGGCTTGAAAGCTCCCACG-3′ | 50 |
| 2 | 8xFN3_rev | | |
| miniUSH2A- | Ss-8xFN3 | 5′-CTTTCAAGCCGAGCCGAGAAGTG-3′ | 51 |
| 2 | fwd | | |
| miniUSH2A- | 8xFN3-TM- | 5′-TCGGAAGCCCACAGACTCTCCAC-3′ | 52 |
| 2 | end rev | | |
| miniUSH2A- | 8xFN3-TM- | 5′-GTCTGTGGGCTTCCGAGTGGATC-3′ | 53 |
| 2 | end fwd | | |
| miniUSH2A- | TM-end- | 5′-GCCAAGCTTGCATGCCTTACAGGTGGGTGTCT-3′ | 54 |
| 2 | pUC19L rev | | |
| miniUSH2A- | Gateway | 5′-GGGGACAAGTTTGTACAAAAAAGCAGGCTTCGCCGC | 55 |
| 2 | cloning | CGCCATGAATTGCCCAGTTCTTTC-3′ | |
| | fwd | | |
| miniUSH2A- | Gateway | 5′-GGGGACCACTTTGTACAAGAAAGCTGGGTCTTACAG | 56 |
| 2 | cloning | GTGGGTGTCTGT-3′ | |
| | rev | | |
| miniUSH2A- | pUC19L- | 5′-AATTCGAGCTCGGTACATGAATTGCCCAGTTCT-3′ | 57 |
| 1 | ss_fwd | | |
| miniUSH2A- | Ss_4xEGF- | 5′-GGATTGTAACATCCAACATCATTAAAGC-3′ | 58 |
| 1 | lamG rev | | |
| miniUSH2A- | 4xEGF-LamG | 5′-TTGGATGTTACAATCCGTCAGCTATTT-3′ | 59 |
| 1 | fwd | | |
| miniUSH2A- | LamG-8xFN3 | 5′-CGGCTCGGACCCCGTGTAAATTTAAC-3′ | 60 |
| 1 | rev | | |
| miniUSH2A- | 8xFN3-TM- | 5′-CACGGGGTCCGAGCCGAGAAGTG-3′ | 61 |
| 1 | end fwd | | |
| miniUSH2A- | TM-end- | 5′-GCCAAGCTTGCATGCCTTACAGGTGGGTGTCT-3′ | 62 |
| 1 | pUC19L rev | | |
| miniUSH2A- | Gateway | 5′-GGGGACAAGTTTGTACAAAAAAGCAGGCTTCGCCGC | 63 |
| 1 | cloning | CGCCATGAATTGCCCAGTTCTTTC-3′ | |
| | fwd | | |
| miniUSH2A- | Gateway | 5′-GGGGACCACTTTGTACAAGAAAGCTGGGTCTTACAG | 64 |
| 1 | cloning | GTGGGTGTCTGT-3′ | |
| | rev | | |
| miniUSH2A- | fwd | 5′-AGACACTCTGCAGTATTCAC-3′ | 65 |
| 1 and -2 | | | |
| detection | | | |
| miniUSH2A- | rev | 5′-CAGAACTGAATACTTTCAGC-3′ | 66 |
| 1 detection | | | |
| miniUSH2A- | rev | 5′-GAGTCGTTTGAGGTAGCAGA-3′ | 67 |
| 2 detection | | | |
| miniUSH2A- | fwd | 5′-TGCCTCGTTTCTTCACAGTC-3′ | 68 |
| 1 and -2 | | | |
| qPCR and | | | |
| miniUSH2A- | | | |
| 5 and -6 | | | |
| detection | | | |
| miniUSH2A- | rev | 5′-GAGCCCAATGAAAGAACTGG-3′ | 69 |
| 1 and -2 | | | |
| qPCR and | | | |
| miniUSH2A- | | | |
| 5 and -6 | | | |
| detection | | | |
| gusb qPCR | fwd | 5′-GTCGTCCCGTCACATTTATTAC-3′ | 70 |
| gusb qPCR | rev | 5′-ATCATGCAGTCCTACTCTGACAC-3′ | 71 |
| miniUSH2A- | pUC19L- | 5′-AATTCGAGCTCGGTACATGAATTGCCCAGTTCT-3′ | 76 |
| 6 | ss_fwd | | |
| miniUSH2A- | Ss- | 5′-CCTTTGCTCTTGAAAGCTCCCACG-3′ | 77 |
| 6 | 1xFN3_rev | | |
| miniUSH2A- | Ss-1xFN3- | 5′-CTTTCAAGAGCAAAGGACCGACA-3′ | 78 |
| 6 | TM-end_fwd | | |
| miniUSH2A- | TM-end- | 5′-GCCAAGCTTGCATGCCTTACAGGTGGGTGTCT-3′ | 79 |
| 6 | pUC19L_rev | | |
| miniUSH2A- | Gateway | 5′-GGGGACAAGTTTGTACAAAAAAGCAGGCTTCGCCGC | 80 |
| 6 | cloning | CGCCATGAATTGCCCAGTTCTTTC-3′ | |
| | fwd | | |
| miniUSH2A- | Gateway | 5′-GGGGACCACTTTGTACAAGAAAGCTGGGTCTTACAG | 81 |
| 6 | cloning | GTGGGTGTCTGT-3′ | |
| | rev | | |
| miniUSH2A- | pUC19L- | 5′-AATTCGAGCTCGGTACATGAATTGCCCAGTTCT-3′ | 82 |
| 5 | ss_fwd | | |
| miniUSH2A- | Ss-TM- | 5′-TCGGAAGCCTTGAAAGCTCCCACG-3′ | 83 |
| 5 | end_rev | | |
| miniUSH2A- | Ss-TM- | 5′-CTTTCAAGGCTTCCGAGTGGATC-3′ | 84 |
| 5 | end_fwd | | |
| miniUSH2A- | TM-end- | 5′-GCCAAGCTTGCATGCCTTACAGGTGGGTGTCT-3′ | 85 |
| 5 | pUC19L_rev | | |
| miniUSH2A- | Gateway | 5′-GGGGACAAGTTTGTACAAAAAAGCAGGCTTCGCCGC | 86 |
| 5 | cloning | CGCCATGAATTGCCCAGTTCTTTC-3′ | |
| | fwd | | |
| miniUSH2A- | Gateway | 5′-GGGGACCACTTTGTACAAGAAAGCTGGGTCTTACAG | 87 |
| 5 | cloning | GTGGGTGTCTGT-3′ | |
| | rev | | |

Generation of Tol2 Transposase mRNA

Transposase mRNA was generated using the pCS2FA-transposase plasmid as a template. After a phenol:chloroform extraction, the vector was linearized using Not1 (NEB, #R0189S), and subsequently purified with DNA clean & Concentrator™ 5-kit (Zymo Research, #D4003T). Capped RNA synthesis was performed using the mMESSAGE mMACHINE™ SP6 Transcription Kit (ThermoFisher, #AM1340) according to manufacturer's protocol. Obtained transcripts were purified using the NucleoSpin® RNA kit (MACHEREY-NAGEL, #740955.250).

Micro-Injections

Zebrafish eggs were obtained from natural spawning. 1 nl of a mixture containing Tol2 transposase mRNA (250 ng/ul), miniUSH2A expression construct (250 ng/ul), KCL (0.2 M) and phenol red (0.05%) was injected into 1-cell-stage embryos of the ush2a^{rmc1} line using a Pneumatic PicoPump pv280 (World Precision Instruments). After injection, embryos were raised at 28° C. in E3 embryo medium (5 mM NaCl, 0.17 mM KCl, 0.33 mM CaCl2), 0.33 mM MgSO4) supplemented with 0.1% v/v methylene blue. At 4 days post fertilization (dpf), embryos were selected for heart-specific EGFP expression. EGFP-positive larvae were raised and subsequently outcrossed with homozygous ush2a^{rmc1} mutants to determine germline transmission of the miniUSH2A gene.

Genotyping Transgenic miniUSH2A Zebrafish

Genomic DNA was isolated from 5 pooled EGFP-positive larvae after a two hour incubation step at 55° C. in lysis buffer (10 mM Tris-HCl pH 8.2, 10 mM EDTA, 100 mM NaCl, 0.5% SDS) supplemented with freshly added proteinase K to a final concentration of 0.20 mg/ml (Invitrogen, #25530049). Isolated genomic DNA (40 ng) was used as input in a PCR to detect miniUSH2A-1,-2, -5 and -6. For this purpose, the Phusion® High-Fidelity PCR Kit (New England Biolabs, E0553) with forward primer SEQ ID NO: 65 5′-AGACACTCTGCAGTATTCAC-3′ (3xPRE-ZOP promoter) and reverse primer SEQ ID NO: 66 5′-CAGAACTGAATACTTTCAGC-3′ (miniUSH2A-1), SEQ ID NO: 67 5′-GAGTCGTTTGAGGTAGCAGA-3′ (miniUSH2A-2), and forward primer SEQ ID NO: 68 5′-TGCCTCGTTTCTTCACAGTC-3′ with reverse primer SEQ ID NO: 69 5′-GAGCCCAATGAAAGAACTGG-3′ (miniUSH2A-5 and -6) were employed. The cycling conditions were as follows: 98° C. 60 seconds, 30 cycles of 98° C. 10 seconds, 56° C. 30 seconds, and 72° C. 30 seconds and a final 72° C. 5 minutes. Amplified fragments were gel-extracted using the NucleoSpin® Gel and PCR Clean-up kit (MACHERY-NAGEL, #740609.250) and sequence verified.

Immunohistochemistry

Zebrafish larvae (4-6 dpf) were positioned (ventral side downwards) in Tissue-Tek (4583, Sakura), frozen in melting isopentane and cryosectioned following standard protocols (7 μm thickness along the lens/optic nerve axis). Sections were permeabilized using 0.01% Tween-20 in PBS followed by a blocking step using blocking solution (10% normal goat serum, 2% BSA in PBS). Primary antibodies diluted in blocking solution were incubated overnight at 4° C. The following primary antibodies were used: mouse anti-usherin-C (1:100; used for detection of miniUSH2A-5 and -6 (FIG. 3B)), rabbit anti-poc5 (1:500; Bethyl Laboratories, #BET A303-341A), rabbit anti-zebrafish Whrnb (1:300; Novus Biological, #42690002), rabbit anti-zebrafish usherin-C (1:500; Novus Biological, #27640002), and mouse anti-centrin (1:500; Novus Biological, #2712468/2677126). The secondary antibodies were goat anti-mouse Alexa Fluor 488 or 568 and goat anti-rabbit Alexa Fluor 488 or 568 (1:800, Molecular Probes-Invitrogen Carlsbad, Calif., USA), diluted in blocking buffer supplemented with DAPI (1:8000) and incubated for 1 hour. Sections were post-fixed in 4% PFA for 5-10 minutes and embedded with Prolong Gold Anti-fade (Thermo Fisher). For the immunofluorescence analyses using rabbit anti-human usherin-C (1:500, kindly provided by Prof. Dr. D. Cosgrove; Zallocchi et al, 2010; used for detection of miniUSH2A-1 and -2 (FIG. 3A)), two adaptations to the protocol were made. The sections were permeabilized in PBS with 0.1% Triton-X-100 for 20 minutes and the used blocking solution consisted of 10% normal goat serum, 2% BSA, 0.1% Triton-X-100 in PBS. Images were taken with an Axioplan2 Imaging fluorescence microscope (Zeiss) equipped with a DC350FX camera (Zeiss, Germany). For quantification of fluorescence after anti-Whrnb and anti-usherin labelings, microscope sections were analyzed using ImageJ. The region of interest was determined in the Alexa Fluor 488 (anti-centrin signal) channel using the “find maxima” option. The 488 channel layer was projected onto the Alexa Fluor 568 (anti-Whrnb or anti-usherin signal) channel after using the ‘substract background’ function. Next, the ‘set measurements’, ‘analyze particles’ and ‘measure’ tools were used in the Alexa Fluor 568 channel, respectively, to determine the mean gray intensity. P-values were calculated using a two-tailed unpaired Student's t-test.

Genomic qPCR Analysis

Genomic DNA was isolated from single larvae or adult zebrafish finclips using the QIAamp DNA Mini Kit (Qiagen, #51304) following the manufacturer's protocol. Genomic qPCRs were performed to quantify copy numbers of miniUSH2A-1 and -2 using 6 ng genomic DNA as input. Specific primers were designed with Primer3Plus (fwd=SEQ ID NO: 68, 5′-TGCCTCGTTTCTTCACAGTC-3′ and rev=SEQ ID NO: 69, 5′-GAGCCCAATGAAAGAACTGG-3′) covering the transition between the opsin promoter and the start of both miniUSH2A-1 and -2. As an internal reference gene gusb (ENSDART00000091932.5) was employed using fwd=SEQ ID NO: 70, 5′-GTCGTCCCGTCACATTTATTAC-3′ and rev=SEQ ID NO: 71, 5′-ATCATGCAGTCCTACTCTGACAC-3′. All reaction mixtures were prepared with the GoTaq qPCR Master Mix (Promega A6001) in accordance with the manufacturer's protocol. All reactions were performed in triplicate with the Applied Biosystems Fast 7900 system. MiniUSH2A/gusb ratios were calculated using the ΔCt method to obtain relative miniUSH2A copy number.

Adaptor-Ligation PCR

To determine the genomic integration sites of miniUSH2A-1 and -2 and validate the numbers of genomic insertions an adaptor-ligation PCR strategy was used, as previously described (Suster et al, 2009). As input ˜150 ng of genomic DNA extracted from single larvae was used. Amplified fragments were gel-extracted using the NucleoSpin® Gel and PCR Clean-up kit (MACHERY-NAGEL, #740609.250) and sequence verified.

GST Pull-Down

In order to produce GST (glutathione S-transferase) fusion proteins, Escherichia coli BL21-DE3 was transformed with plasmid pDEST15-usherin_icd (aa 5064-5202). After induction with IPTG, GST fusion proteins were isolated as described before (Van Wijk et al, 2006). HA-tagged Whrna was produced by transfecting HEK293T cells with pcDNA3-HA-Whrna, using the transfection reagent polyethylenimine (PEI, PolySciences), according to the manufacturer's instructions. Twenty-four hours after transfection, cells were washed with PBS and subsequently lysed on ice using lysisbuffer (50 mM Tris-HCL pH7.5, 150 mM NaCl, 0.5% Triton-X-100) supplemented with Complete protease inhibitor cocktail (Roche, Germany). GST pull-down assays were performed as described previously (Van Wijk et al, 2006). Proteins were resolved on 4-12% NuPage gradient gels (Thermo Fisher #NP0321 BOX) and analyzed on immunoblots. Bands were visualized by using the Odyssey Infrared Imaging System (LI-COR, USA). HA-tagged Whrna was detected by anti-HA monoclonal antibodies (Sigma, #H9658). As secondary antibody, Alexa Fluor 680 goat-anti-rabbit IgG was used (Molecular Probes, USA).

Visual Motor Response Assay

Locomotor activity was tracked and analyzed using EthoVision XT 11.0 software (Noldus Information Technology BV, Wageningen, The Netherlands). Larvae (5dpf) were individually positioned into a 48-wells plate, containing 200 μl of E3 medium per well. The 48-wells plate was placed in the observation chamber of the DanioVision™ tracking system (Noldus Information Technology BV, Wageningen, The Netherlands). After 20 minutes of dark adaption, the larvae were exposed to 3 cycles of 10 minutes dark/10 minutes light. In all experiments, larvae were subjected to locomotion analyses between 13:00-18:00 in a sound- and temperature-controlled (28° C.) behavioral testing room.

Electroretinograms

ERG measurements were performed on isolated larval eyes (5-7 dpf) as previously described (Sirisi et al, 2014). Larvae were dark-adapted for a minimum of 30 min prior to the measurements and subsequently handled under dim red illumination. Isolated eyes were positioned to face the light source. Under visual control via a standard microscope equipped with red illumination (Stemi 2000C, Zeiss, Oberkochen, Germany), the recording electrode with an opening of approximately 20 μm at the tip was placed against the center of the cornea. This electrode was filled with E3 medium (5 mM NaCl, 0.17 mM KCl, 0.33 mM CaCl₂), and 0.33 mM MgSO4). The electrode was moved with a micromanipulator (M330R, World Precision Instruments Inc., Sarasota, USA). A custom-made stimulator was invoked to provide light pulses of 100 ms duration, with a light intensity of 6000 lux. For the light pulses a ZEISS XBO 75W light source was employed and a fast shutter (Uni-Blitz Model D122, Vincent Associates, Rochester, N.Y., USA) driven by a delay unit interfaced to the main ERG recording setup. Electronic signals were amplified 1000 times by a pre-amplifier (P55 A.C. Preamplifier, Astro-Med. Inc, Grass Technology) with a band pass between 0.1 and 100 Hz, digitized by DAQ Board NI PCI-6035E (National Instruments) via NI BNC-2090 accessories and displayed via a self-developed NI Labview program (Rinner et al, 2005). Statistical analyses were performed using SPSS Statistics 22 (IBM), and graphs were generated in Excel (Microsoft). Statistical significance was set at p<0.05. All experiments were performed at room temperature (22° C.).

Results

Considering the transgene packaging capacity of the conventional LV and AAV vectors (Lopes et al, 2013), we constructed four human USH2A minigenes (FIG. 1A). MiniUSH2A-1 (˜6.8 kb) encodes a polypeptide of 2,262 amino acids containing the signal sequence (S), the laminin G-like domain (LamGL), the laminin N-terminal domain (LamNT), four EGF Lam domains, one LamG domain, the cysteine-rich region flanked by two and five FN3 domains at the N- and C-terminal side respectively, the transmembrane domain (TM) and the intracellular region containing the class I PDZ-binding motif (PBM). MiniUSH2A-2 (˜4.1 kb) encodes a polypeptide of 1,375 amino acids that contains the usherin signal sequence (S), two FN3 domains, the cysteine-rich region, five additional FN3 domains, the transmembrane domain (TM) and the intracellular region containing the class I PDZ-binding motif (PBM). MiniUSH2A-6 (˜1.3 kb) encodes a polypeptide of 435 amino acids containing the signal sequence (S), one FN3 domain, the transmembrane domain (TM) and the intracellular region containing the class I PDZ-binding motif (PBM). MiniUSH2A-5 (˜1 kb) encodes a polypeptide of 331 amino acids containing the signal sequence (S), the transmembrane domain (TM) and the intracellular region containing the class I PDZ-binding motif (PBM). We cloned the coding sequences of miniUSH2A-1, -2, -5 and -6 in the Tol2 transposon vector pDestTol2CG2, between an enhanced zebrafish opsin promoter and the internal ribosomal entry site (IRES) EGFP. This vector further contains the coding sequences of EGFP under the control of a heart-specific cmc12 promoter (FIGS. 1B and C). The complete expression cassette was flanked by Tol2 sites.

MiniUSH2A-1 and miniUSH2A-2 Insertion into the Genome of ush2a^{rmc1} Zebrafish

We injected the minigene-containing vectors together with Tol2 transposase mRNA into homozygous one-cell staged ush2a^{rmc1} embryos (FIGS. 1D and E). ush2a^{rmc1} mutants contain a frameshift-inducing mutation in ush2a exon 13 (c.2337_2344delinsAC; p.Cys780 GlnfsTer32) that leads to a premature termination of translation and, as a consequence, absence of zebrafish usherin. Injected larvae (F0) that were positive for heart-specific EGFP expression at 4 dpf were raised and outcrossed with homozygous ush2a^{rmc1} fish in order to test for germline transmission of the miniUSH2A expression cassettes. Again, larvae (F1) with heart-specific EGFP expression were selected. Tol2 transposase induces a random integration of (multiple) transposable elements into the genome. Therefore we performed a genomic qPCR analysis to determine the number of miniUSH2A-1 and -2 copies that were integrated in the genome of the transgenic F1 larvae. This revealed that for both USH2A minigenes multiple copies were present in the genomes of F1 larvae. The same analyses were performed after a second outcross with ush2a^{rmc1} mutants. For both minigenes F2 larvae were identified with a single copy minigene insertion. This was corroborated by an adaptor ligation assay. This assay also revealed the exact genomic position of minigene insertions. Single copies of miniUSH2A-1 were found to be integrated at two distinct genomic loci: an intergenic region on chromosome 18 and the zinc-finger CCCH-type containing 4 (zc3h4) gene on chromosome 15 (FIG. 2A). So far, ZC3H4 mutations have not been associated with a human disease and also no animal models for ZC3H4 are available. Deletion of ZC3H4 in patients with the 19q13.32 microdeletion syndrome has also not been reported to be associated with retinal dysfunction (Travan, 2017). MiniUSH2A-2 was found to be present as a single copy integration in chromosome 17, thereby disrupting the zgc:154061 gene (FIG. 2B). Mutations of C15ORF41, the human ortholog of zgc:154061, are associated with congenital dyserythropoietic anemia (OMIM: 615631), an inherited disorder that affects the development of red blood cells. Although no retinal phenotype has been described to be associated with C15ORF41 or ZC3H4 mutations, we questioned whether disruption of these genes due to the integration of an USH2A minigene would affect retinal morphology.

MiniUSH2A-1, -2, -5 and -6 are Expressed and Localize to the Photoreceptor Periciliary Region

We first determined whether the USH2A minigenes are expressed in photoreceptor cells and whether they localize to the photoreceptor periciliary region in transgenic zebrafish larvae. For this purpose, we performed immunofluorescence assays with an antibody that specifically recognizes human usherin. As expected, no anti-usherin signal was observed in retina of wild-type and ush2a^{rmc1} larvae (FIG. 3A D and 3A E). In the retina of transgenic larvae, miniUSH2A-1 and -2 were detected adjacent to the connecting cilium and the basal body as marked by anti-centrin (FIG. 3A B and 3A C). MiniUSH2A-5 and -6 were also expressed and detected adjacent to basal body and connecting cilium marker poc5 (FIG. 3B B and 3B C). We next assessed whether the expression of the miniUSH2A genes had an adverse effect on retinal morphology. Histological analysis of transgenic fish expressing miniUSH2A showed a normal retinal lamination and cellular organization in both larvae and adults as compared to wild-type controls (5 dpf: n=21; 6 months post fertizilization (mpf) n=2). Also, no other abnormalities in overall body morphology or swimming behavior were observed. Therefore, we conclude that the genomic integration and expression of miniUSH2A-1, -2, -5 or -6 has no gross negative consequences for zebrafish development and functioning of adult fish in the presented transgenic zebrafish lines.

Expression of miniUSH2A Restores Whrna Levels at the Photoreceptor Periciliary Region

Usherin and whirlin interact and are mutually dependent on each other for their localization at the photoreceptor periciliary membrane (Van Wijk et al, 2006; Yang et al, 2010; Dona et al, submitted). Therefore, we questioned whether the expression of miniUSH2A-1 or -2 would result in the restoration of Whrna localization in ush2a^{rmc1} zebrafish photoreceptor cells. We first confirmed that the intracellular region of human usherin and zebrafish Whrna indeed interact. In a glutathione S-transferase (GST) pull-down assay, full length HA-tagged Whrna was pulled down from HEK293T cell lysates by GST-fused usherin aa 5064-5202 but not by GST alone (FIG. 4C). Subsequently, we performed immunohistochemistry using anti-Whrna antibodies. Anti-centrin antibodies were employed as a marker for the basal body and connecting cilium. In transgenic larvae expressing miniUSH2A-1 or -2, Whrna levels at the photoreceptor periciliary regions were significantly increased as compared to those in ush2a^{rmc1} larvae (FIGS. 4A and 4B). This demonstrates that expression of miniUSH2A-1 and miniUSH2A-2 leads to an USH2A-Whrna complex at the photoreceptor periciliary region, potentially resulting in the (partial) functional rescue.

Expression of miniUSH2A Rescues the Visual Motor Response

The next step was to assess whether supplementing ush2a^{rmc1} zebrafish with human miniUSH2A-1 or -2 (partially) restores retinal function. As shown before, the visual motor response (VMR) is a semi high-throughput behavioral assay by which defects in visual function can be detected in a sensitive and robust way. We demonstrated that ush2a^{rmc1} larvae have a decreased light-ON VMR as compared to wild-type controls (FIG. 5). Recording the light-ON VMR of transgenic miniUSH2A-1 or -2 ush2a^{rmc1} larvae demonstrated that expression of either miniUSH2A protein restored the VMR. Subsequently, we performed quantitative two-sample Hotelling's T-squared tests for the pairwise comparison of the different conditions (Liu et al, 2015). The maximum velocity during the first 2 seconds after the light-ON stimulus, which is regarded to be the eye-specific response, was significantly improved in ush2a^{rmc1} larvae expressing miniUSH2A-1 or -2 as compared to ush2a^{rmc1} mutant larvae. Furthermore, the recorded VMRs in transgenic miniUSH2A-1 or -2 transgenic larvae was not significantly different from the VMR recorded in age-matched wild-type larvae (FIG. 5).

MiniUSH2A Expression Enhances b-Wave Amplitudes of the Electroretinogram

We next recorded electroretinograms (ERGs) to determine the functionality of the retina of transgenic larvae expressing miniUSH2A-1, -2, -5 and -6 (5 dpf). Average ERGs from dark-adapted individual wild-type, ush2a^{rmc1}, miniUSH2A-1 and miniUSH2A-2 larvae are shown in FIG. 6A_A, together with the maximum average amplitudes plotted as bar-graphs (FIG. 6A_B). Analysis of retinal function by ERG revealed a significant improvement of the b-wave amplitudes of the miniUSH2A-1 (37%) and -2 (57%) expressing larvae at 5dpf compared to the ush2a^{rmc1} larvae (FIG. 6A). Statistical analyses revealed no significant differences in b-wave amplitudes recorded in ush2a^{rmc1} larvae expression miniUSH2A-1 or -2. Also the b-wave amplitudes of wild-type control larvae and larvae expressing the miniUSH2A-1 gene were not significantly different. Average ERGs from dark-adapted miniUSH2A-6 (FIG. 6B_A) and miniUSH2A-5 (FIG. 6B_C) larvae are shown in FIG. 6A_A, together with the maximum average b wave amplitudes per individual larva plotted as dot plots (FIGS. 6A_B and D; n˜10 larvae). As a negative control, GFP negative larvae were used from the same miniUSH2A-5 or -6 clutch. A clear trend was observed in improvement of the b wave amplitudes recorded in both miniUSH2A-5 and -6 expressing transgenic larvae as compared to clutch-matched GFP negative mutant larvae.

Overall, our results demonstrate that the expression of minigenes according to the invention, as exemplified by miniUSH2A-1, -2, -5 and -6, improves retinal function of ush2a^{rmc1} larvae. This suggests that the minigenes according to the invention can successfully be used in the treatment of human subjects, either by itself or in a vector such as state of the art adeno associated vectors.

### REFERENCES

Kimberling, W. J., Hildebrand, M. S., Shearer, A. E., Jensen, M. L., Halder, J. A., Trzupek, K., Cohn, E. S., Weleber, R. G., Stone, E. M. & Smith, R. J. Frequency of Usher syndrome in two pediatric populations: Implications for genetic screening of deaf and hard of hearing children. Genet Med. 12, 512-516. doi: 10.1097/GIM.0b013e3181e5afb8 (2010).
Hartong, D. T., Berson, E. L. & Dryja, T. P. Retinitis pigmentosa. Lancet 368, 1795-1809, doi:50140-6736(06)69740-7 (2006).
McGee, T. L., Seyedahmadi, B. J., Sweeney, M. O., Dryja, T. P. & Berson, E. L. Novel mutations in the long isoform of the USH2A gene in patients with Usher syndrome type II or non-syndromic retinitis pigmentosa. J Med Genet 47, 499-506, doi:10.1136/jmg.2009.075143 (2010).
Bainbridge, J. W. et al. Effect of gene therapy on visual function in Leber's congenital amaurosis. N Engl J Med 358, 2231-2239, doi:10.1056/NEJMoa0802268 (2008).
Cideciyan, A. V., Aleman, T. S., Boye, S. L., Schwartz, S. B., Kaushal, S., Roman, A. J., Pang, J. J., Sumaroka, A., Windsor, E. A., Wilson, J. M., Flotte, T. R., Fishman, G. A., Heon, E., Stone, E. M., Byrne, B. J., Jacobson, S. G., Hauswirth, W. W. Human gene therapy for RPE65 isomerase deficiency activates the retinoid cycle of vision but with slow rod kinetics. Proc Natl Acad Sci USA. 105, 15112-15117. doi: 10.1073/pnas.0807027105 (2008)
Hauswirth, W. W., Aleman, T. S., Kaushal, S., Cideciyan, A. V., Schwartz, S. B., Wang, L., Conlon, T. J., Boye, S. L., Flotte, T. R., Byrne, B. J. & Jacobson, S. G. Treatment of leber congenital amaurosis due to RPE65 mutations by ocular subretinal injection of adeno-associated virus gene vector: short-term results of a phase I trial. Hum Gene Ther. 19, 979-990. doi: 10.1089/hum.2008.107 (2008).
Maguire, A. M., Simonelli, F., Pierce, E. A., Pugh, E. N. Jr., Mingozzi, F., Bennicelli, J., Banfi, S., Marshall, K. A., Testa, F., Surace, E. M., Rossi, S., Lyubarsky, A., Arruda, V. R., Konkle, B., Stone, E., Sun, J., Jacobs, J., Dell'Osso, L., Hertle, R., Ma, J. X., Redmond, T. M., Zhu, X., Hauck, B., Zelenaia, O., Shindler, K. S., Maguire, M. G., Wright, J. F., Volpe, N.J., McDonnell, J. W., Auricchio, A., High, K. A. & Bennett, J. Safety and efficacy of gene transfer for Leber's congenital amaurosis. N Engl J Med. 358, 2240-2248. doi: 10.1056/NEJMoa0802315 (2008).
Hashimoto, T. et al. Lentiviral gene replacement therapy of retinas in a mouse model for Usher syndrome type 1B. Gene Ther 14, 584-594, doi:10.1038/sj. gt.3302897 (2007).
Lopes, V. S. et al. Retinal gene therapy with a large MYO7A cDNA using adeno-associated virus. Gene Ther 20, 824-833, doi:10.1038/gt.2013.3 (2013).
Colella, P. et al. Efficient gene delivery to the cone-enriched pig retina by dual AAV vectors. Gene Ther 21, 450-456, doi:10.1038/gt.2014.8 (2014).
Zallocchi, M. et al. EIAV-based retinal gene therapy in the shaker1 mouse model for usher syndrome type 1B: development of UshStat. PLoS One 9, e94272, doi:10.1371/journal.pone.0094272 (2014).
Kimmel, C. B., Ballard, W. W., Kimmel, S. R., Ullmann, B. & Schilling, T. F. Stages of embryonic development of the zebrafish. Developmental dynamics: an official publication of the American Association of Anatomists 203, 253-310, doi:10.1002/aja.1002030302 (1995).
Kennedy, B. N., Vihtelic, T. S., Checkley, L., Vaughan, K. T. & Hyde, D. R. Isolation of a zebrafish rod opsin promoter to generate a transgenic zebrafish line expressing enhanced green fluorescent protein in rod photoreceptors. J Biol Chem 276, 14037-14043, doi:10.1074/jbc. M010490200 (2001).
Kwan, K. M. et al. The Tol2kit: a multisite gateway-based construction kit for Tol2 transposon transgenesis constructs. Dev Dyn 236, 3088-3099, doi:10.1002/dvdy.21343 (2007).
Zallocchi, M., Sisson, J. H. & Cosgrove, D. Biochemical characterization of native Usher protein complexes from a vesicular subfraction of tracheal epithelial cells. Biochemistry 49, 1236-1247, doi:10.1021/bi9020617 (2010).
van Wijk, E. et al. The DFNB31 gene product whirlin connects to the Usher protein network in the cochlea and retina by direct association with USH2A and VLGR1. Human molecular genetics 15, 751-765, doi:10.1093/hmg/ddi490 (2006).
Suster, M. L., Kikuta, H., Urasaki, A., Asakawa, K. & Kawakami, K. Transgenesis in zebrafish with the tol2 transposon system. Methods in molecular biology 561, 41-63, doi:10.1007/978-1-60327-019-9_3 (2009).
Sirisi, S. et al. Megalencephalic leukoencephalopathy with subcortical cysts protein 1 regulates glial surface localization of GLIALCAM from fish to humans. Human molecular genetics 23, 5069-5086, doi:10.1093/hmg/ddu231 (2014).
Rinner, O., Makhankov, Y. V., Biehlmaier, O. & Neuhauss, S. C. Knockdown of cone-specific kinase GRK7 in larval zebrafish leads to impaired cone response recovery and delayed dark adaptation. Neuron 47, 231-242, doi:10.1016/j.neuron.2005.06.010 (2005).
Lopes, V. S. et al. Retinal gene therapy with a large MYO7A cDNA using adeno-associated virus. Gene Ther 20, 824-833, doi:10.1038/gt.2013.3 (2013).
Travan, L. et al. Phenotypic expression of 19q13.32 microdeletions: Report of a new patient and review of the literature. Am J Med Genet A, doi:10.1002/ajmg.a.38256 (2017).
Yang, J. et al. Ablation of whirlin long isoform disrupts the USH2 protein complex and causes vision and hearing loss. PLoS genetics 6, e1000955, doi:10.1371/journal.pgen.1000955 (2010). Liu, Y. et al. Statistical Analysis of Zebrafish Locomotor Response. PLoS One 10, e0139521, doi:10.1371/journal.pone.0139521 (2015).

1. A polynucleotide construct comprising:
a signal sequence, preferably an USH2A signal sequence,
a polynucleotide encoding an USH2A transmembrane domain (TM),
a polynucleotide encoding an USH2A intracellular region including the PDZ binding motif (PBM).

2. The polynucleotide construct according to claim 1, further comprising a polynucleotide encoding an USH2A fibronectin 3 domain (FN3).

3. The polynucleotide construct according to claim 1, further comprising a polynucleotide encoding an USH2A cysteine-rich fibronectin 3 domain.

4. The polynucleotide construct according to claim 3, comprising at least two polynucleotides encoding an USH2A fibronectin 3 domain (FN3).

5. The polynucleotide construct according to claim 4, comprising at least seven polynucleotides encoding an USH2A fibronectin 3 domain (FN3).

6. The polynucleotide construct according to claim 1, further comprising a polynucleotide encoding a domain selected from the group consisting of:
a polynucleotide encoding an USH2A laminin G-like domain (LamGL), a polynucleotide encoding an USH2A laminin N-terminal domain (LamNT), a polynucleotide encoding an USH2A laminin-type EGF-like domain (EGF Lam) and a polynucleotide encoding an USH2A laminin G domain (LamG).

7. The polynucleotide construct according to claim 5, further comprising a polynucleotide encoding an USH2A laminin G-like domain (LamGL), a polynucleotide encoding an USH2A laminin N-terminal domain (LamNT), at least four polynucleotides encoding an USH2A laminin-type EGF-like domain (EGF Lam), and an USH2A polynucleotide encoding a laminin G domain (LamG).

8. The polynucleotide construct according to claim 1, wherein the polynucleotide construct has at least 50% sequence identity with SEQ ID NO: 40, 42, 44, 46, 48, 75 or wherein the polynucleotide construct encodes a protein having at least 50% sequence identity with SEQ ID NO: 39, 41, 43, 45, 47, 74.

9. The polynucleotide construct according to claim 1, further comprising regulatory sequences that direct expression of the coding sequences in the polynucleotide construct.

10. (canceled)

11. A vector comprising the polynucleotide construct according to claim 1.

12.-14. (canceled)

15. A method of treatment or prevention of USH2A-associated retinitis pigmentosa in a subject in need thereof, comprising administration of the polynucleotide construct according to claim 1.

16.-17. (canceled)

18. The vector according to claim 11, wherein the vector is an adeno-associated viral vector (AAV).

19. The vector according to claim 19, wherein the AAV further comprises an AAV inverted terminal repeat.

20. The vector according to claim 11, wherein the vector is a lentiviral vector (LV).

21. The vector according to claim 20, wherein the LV further comprises an LV long terminal repeat (LTR).
